(19) United States
(12) Patent Application Publication
(10) Pub. No.: US 2026/0351337 A1
(43) Pub. Date: Nov. 5, 2026

(54) METHOD OF PREDICTION OF LIGAND-PROTEIN BINDING AFFINITIES USING META-MODELS

(71) Applicant: Yale University, New Haven, CT (US)

(72) Inventors: Ho-Joon Lee, New Haven, CT (US); Prashant Siva Emani, New Haven, CT (US)

(21) Appl. No.: 19/488,389

(22) PCT Filed: May 30, 2024
(86) PCT No.: PCT/US2024/031704

§ 371 Date: Nov. 26, 2025

Related U.S. Application Data
(60) Provisional application No. 63/504,945, filed on May 30, 2023.

(51) Int. Cl. G16B 15/30; G06N 3/045; G16B 35/20; G16B 40/20

(52) U.S. Cl. CPC G16B 15/30; G06N 3/045; G16B 35/20; G16B 40/20

(57) ABSTRACT

Provided herein are methods of developing a meta-modeling framework for ligand-protein binding affinity prediction by integrating empirical scoring function-based docking and deep learning-based regression outputs through linear and non-linear machine learning models. Thus, empirical docking models and deep learning models were used as base models data. By taking predictions from those base models, machine learning regression models were developed as meta-models. These meta-models significantly improve binding affinity prediction over individual base models and achieve comparable performance to other structure-based deep learning models.

### CROSS-REFERENCE TO RELATED APPLICATIONS

This application claims priority under 35 U.S.C. § 119(e) to U.S. Provisional Patent Application No. 63/504,945, filed May 30, 2023, the disclosure of which is incorporated herein by reference in its entirety.

### SUMMARY OF THE INVENTION

In one aspect, provided herein is a method of training an algorithm to predict ligand-protein binding affinity, the method comprising:
generating at least one docking score for the binding affinity of one or more ligand-protein complexes using one or more structure-based docking tools, wherein the generating of the at least one docking score comprises the steps of:
selecting a receptor structure file;
generating docking poses and associated binding affinity scores for each of the one or more ligand-protein complexes by one or more docking tools;
analyzing the resulting docking poses for the one or more ligand-protein complexes; and
generating at least one deep learning (DL) score for the one or more ligand-protein complexes using one or more sequence-based DL models, wherein the generating of the at least one deep learning score comprises the steps of:
selecting at least one DL model to predict binding affinities of ligand-protein complexes; and
generating scores for the one or more ligand-protein complexes using the at least one DL model;
generating a meta-model training dataset from scores by at least two of docking tools or DL models and at least one physicochemical property of the ligand or the protein; and
training a machine learning meta-model on the training dataset to generate an algorithm for predicting ligand-protein binding affinities.

In another aspect, the provided herein is an apparatus for predicting a ligand-protein binding affinity, the apparatus comprising: a processor; a memory unit; and a communication interface.

In yet another aspect, provided herein is a non-transitory computer readable storage medium, storing computer-executable instructions for performing the method described elsewhere herein.

In certain embodiments, the generating at least one docking score for the binding affinity comprises employing at least two empirical scoring functions (ESFs) in tandem.

In certain embodiments, the at least two ESFs are SMINA and Vinardo.

In certain embodiments, the method further comprises preprocessing the docking scores for the one or more ligand poses in predicting each ligand-protein complex.

In certain embodiments, the preprocessing comprises filtering the ligand poses based upon the experimental and/or predicted docking poses.

In certain embodiments, the filtering comprises applying a root-mean-squared deviation (RMSD) filter.

In certain embodiments, the filtering comprises at least one of experimental pose filtering or consensus pose filtering.

In certain embodiments, the experimental pose filtering comprises calculating a deviation for at least one of the docking poses predicted by the ESF relative to an experimental structure of the ligand.

In certain embodiments, the consensus pose filtering comprises calculating a deviation between the docking poses predicted by one of the ESFs relative to the docking pose predicted by another one of the ESFs.

In certain embodiments, the filtering cut-off is about 3 Angstrom.

In certain embodiments, selecting the at least one DL model comprises:
inputting ligand-encoding and receptor-encoding embeddings into one or more DL models; determining a performance of the one or more DL models; and selecting the at least one DL model based upon the performance of the one or more DL models.

In certain embodiments, the at least one DL model includes at least one of a pre-trained model or a de novo-trained model.

In certain embodiments, the at least one DL model is either pre-trained or de novo-trained using BindingDB.

In certain embodiments, the at least one DL model is de novo-trained using PDBbind data.

In certain embodiments, the at least one DL model is selected from the group consisting of CNN-CNN, Daylight-AAC, Morgan-AAC, Morgan-CNN, MPNN-CNN, Transformer-CNN, MPNN-Quasi-seq, CNN/RNN-CNN/RNN, CNN-AAC, MPNN-Transformer, MPNN-PseudoAAC, MPNN-AAC, and MPNN-CNN/RNN.

In certain embodiments, the at least one pre-trained DL model is selected from the group consisting of CNN-CNN, Daylight-AAC, Morgan-AAC, Morgan-CNN, MPNN-CNN, and Transformer-CNN.

In certain embodiments, the at least one de novo-trained DL model is selected from the group consisting of CNN-AAC, MPNN-CNN, CNN-CNN, Daylight-CNN, MPNN-AAC, and MPNN-Transformer.

In certain embodiments, the at least one de novo-trained DL model is selected from the group consisting of CNN-AAC, MPNN-CNN, CNN-CNN, Daylight-CNN, MPNN-AAC, MPNN-Transformer, Daylight-AAC, Morgan-AAC, Morgan-CNN, and Transformer-CNN.

In certain embodiments, the at least one pre-trained or the de-novo-trained model is fine-tuned using PDBbind data.

In certain embodiments, the at least one or more DL models are either de novo-trained or fine-tuned using 10 repetitions of 5-fold cross-validations (CVs).

In certain embodiments, principal component analysis (PCA) is performed on the DL scores from all CVs.

In certain embodiments, prediction scores are generated from the first principal components of PCA.

In certain embodiments, the ligand-encoding and the receptor-encoding embeddings is from DeepPurpose library.

In certain embodiments, the meta-model is a linear model or a non-linear model and is selected from the group consisting of LASSO model, ElasticNet model, Linear Regression model, and XGBoost model.

In certain embodiments, the meta-model training dataset further includes molecular weight of the at least two or more ligands.

In certain embodiments, the selecting comprises selecting the DL model that achieves the performance of mean-squared-error (MSE)<0.6.

In certain embodiments, the processor is connected to the memory unit and the communication interface. In certain embodiments, the processor and memory are configured to implement the algorithm trained according to any one of the previous claims.

### BACKGROUND OF THE INVENTION

Molecular interactions such as protein-protein or ligand-protein interactions are fundamental processes in biology as well as biophysics and biochemistry. Knowledge of interaction energy or binding affinity is essential in understanding biological functions and applying to biomedical problems. In drug discovery or development, accurate prediction of binding affinity of a drug ligand bound to a target molecule (typically a protein) is of central importance in identifying the most stable structure of a drug-target complex, i.e., the best 3-dimensional pose (geometry) of a drug in the binding pocket of a target molecule, known as molecular docking. Along with development of molecular docking algorithms, there have been extensive computational studies with decades of efforts to achieve better prediction of ligand-protein binding affinity with applications to high throughput drug screening or virtual screening. This has been achieved by various scoring functions that represent the binding free energy or stability of a ligand-protein complex and select the best docking pose.

Scoring functions were developed traditionally by ab initio physical force field-based, knowledge-based, or empirical methods, or more recently by advanced machine learning or deep learning approaches. The predictive power of traditional or classical scoring functions is known to be very limited due to rigid or naive underlying assumptions, e.g. reliance on linear regression or over-simplification or ignorance of desolvation and entropy contributions because of computational cost. Further, with the groundbreaking advancement of deep learning (DL) in computer science, its applications in biomedicine, including protein structure prediction, binding affinity prediction, and drug discovery, have been very successful and promising. However, in general, DL architectures are highly diverse with a number of hyperparameters such as the network depth and width, loss and activation functions, and a learning rate, which makes model optimization very challenging due to high computational cost. Thus, there is a need in the art to develop better models for ligand-protein binding affinity predictions. The present invention addresses this unmet need.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 shows an overview of workflow according to the embodiments of the present invention.

FIG. 2 illustrates performances of empirical scoring functions and consensus filtering.

FIGS. 3A-3B illustrate performances of deep learning models. (FIG. 3A) Scatter plots of experimental binding affinities (x axis) and model predictions (y axis) for the full 5,305 complexes in the PDBbind Refined Set (266 complexes in the CoreSet benchmark). Each plot shows predictions by the average of the best models or PC1 in terms of Pearson correlation coefficients for the CoreSet in each training strategy. (FIG. 3B) Box-whisker plots of Pearson correlation coefficients (left panel) and mean squared errors (MSE; right panel) for the CoreSet by a total of 22 PDBbind-finetuned (M1 and M2) and PDBbind-trained (M3) models and PC1 models of M1, M2, and M3 along with SMINA and Vinardo models. Predictions of the 22 models are mean values from 10 repeated 5-fold CVs. M1, M2, and M3 correspond to DIF, D2F, and D3 in (FIG. 3A).

FIGS. 4A-4D show model comparison of CoreSet predictions. (FIG. 4A) Heatmap of Pearson correlations between all pairs of CoreSet predictions by the 42 models from the docking and deep learning tools. The dendrograms are results of hierarchical clustering with Euclidean distance and the complete linkage method. (FIG. 4B) Bar plot of Pearson correlations between the experimental binding affinity and predictions by the 42 models for the CoreSet complexes. The models are ordered by the correlation values. (FIG. 4C) Heatmap of absolute errors or deviations between the experimental binding affinity and predictions by the 42 models (columns) for each of the CoreSet complexes (rows). The dendrograms are as in (FIG. 4A). (FIG. 4D) Box-whisker plot of the data used in (FIG. 4B). The models are ordered by the median values.

FIG. 5 shows meta-model predictions by the XGBoost approach for the unfiltered training set. The first row represents the meta-models constructed entirely based on the docking methods, SMINA and Vinardo. The first panel shows results for the two scores alone (E), while the second panel shows the results also including the molecular weight parameter (EW). The remaining rows show results for meta-models that include DL scores. The second, third and fourth rows represent successive processing of the deep learning model scores, with the second row showing the average scores (ED1, ED2, ED3), the third row (if a panel is present) showing the average after fine-tuning (F) using the RefinedSet\CoreSet (ED1-F, ED2-F), and the fourth row showing the scores using the optimal number of PCs from a PCA (P) on the previous level (either fine-tuned or not) (ED1-F-P, ED2-F-P, ED3-P). The columns of the second, third and fourth rows show the three different base training sets used for the DL scores, where the leftmost column is based on the pre-trained models on BindingDB (ED1 series), the central column is based on the de novo-trained models on BindingDB (ED2 series) and the rightmost column is based on the de novo-trained models on PDBbind (ED3 series). The single panel in the last fifth row shows the results for the meta-model ED-A-P, which is based on a PCA on all the training scores from ED1-F, ED2-F, and ED3.

FIG. 6 shows RMSD distributions for the experimental pose-based and consensus pose-based filtering strategies (266 CASF-2016 complexes (upper panel) and Monte Carlo simulations of prediction correlations for the CoreSet 3 Angstrom filtering (lower panel), where the full CoreSet without filtering is subsampled repeatedly down to the number after filtering at the 3 Angstrom level to test whether the observed improvement in correlation with filtering is a random or genuine effect. The dotted lines show the observed prediction correlation from the filtered dataset, and the histograms show the distributions of the 1000 subsamplings.

FIG. 7 shows training and validation of 12 initial BindingDB-trained deep learning models.

FIG. 8 shows regression results of BindingDB Kd for 66,444 ligand-protein complexes by BindingDB-trained, PDBbind-trained, and PDBbind-finetuned models. PDBBind-trained models poorly predict BindingDB Kd, whereas the PDBbind-refined models of the top 6 BindingDB-trained models maintain good prediction of BindingDB Kd.

FIG. 9 shows regression results of PDBbind-v2020-refined by BindingDB pre-trained, BindingDB de novo-trained, PDBbind de novo-trained, and PDBbind-finetuned models.

FIG. 10 shows model comparison of non-core (training set) predictions.

FIG. 11 illustrates feature importance of meta-models.

FIGS. 12A-12D Illustrate prediction synergy and complementarity of docking and DL tools by meta-modeling. (FIG. 12A) Absolute errors (AEs) of the mean predictions for the CASF-2016 benchmark set (266 complexes) by the 4 meta-models with ED-A-P (open circles; ordered by AEs). The blue circles correspond to 104 complexes (39.1%) whose predictions by the DL tools (DAP|PC1 in FIG. 4D) are better than the mean predictions by the 4 meta-models or the 4 docking tools (i.e., lower AEs). The red circles correspond to 39 complexes (14.7%) whose mean predictions by the 4 docking tools are better than those by the meta-models or the DAP|PC1. (FIGS. 12B-12D) Scatter plots of AEs of the (mean) predictions for the CASF-2016 benchmark set by the DAP|PC1 vs. the 4 meta-models (FIG. 12B), by the 4 docking tools vs. the 4 meta-models (FIG. 12C), and by the 4 docking tools vs. the DAP|PC1 (FIG. 12D). The blue and red dots in (FIG. 12B) and (FIG. 12C) correspond to those in (FIG. 12A). In (FIG. 12D), the 48 complexes (18.0%) in red indicate better predictions (i.e., lower AEs) by the docking tools on average, whereas the remaining 218 complexes (82.0%) in blue indicate better predictions by the DAP|PC1.

FIG. 13 shows an overview of workflow with an example meta-model of ED1-F-P.

FIG. 14 is a table showing performance of DL models.

FIG. 15 is a table showing meta-model performances by XGboost.

FIG. 16 is a table showing performance comparisons of a virtual screening benchmark. The best performance value for each target protein is in bold italic, while performance values for models presented herein are in bold. PCC=Pearson correlation coefficient. *Pre-trained on PDBbind v2016 GeneralSet.

FIG. 17 is a table showing dataset sizes for the empirical docking outputs after applying different RMSD filters.

FIG. 18 shows deep learning run times.

FIG. 19 is a table showing meta-model performances version 1.

FIG. 20 is a table showing meta-model performances version 2.

FIG. 21 is a table showing base model score correlations with SwissADME features.

FIG. 22 is a table showing XGBoost meta-model score correlations with SwissADME features.

FIG. 23 is a table showing UniProt and SwissADME enrichment analyses for ligands and proteins.

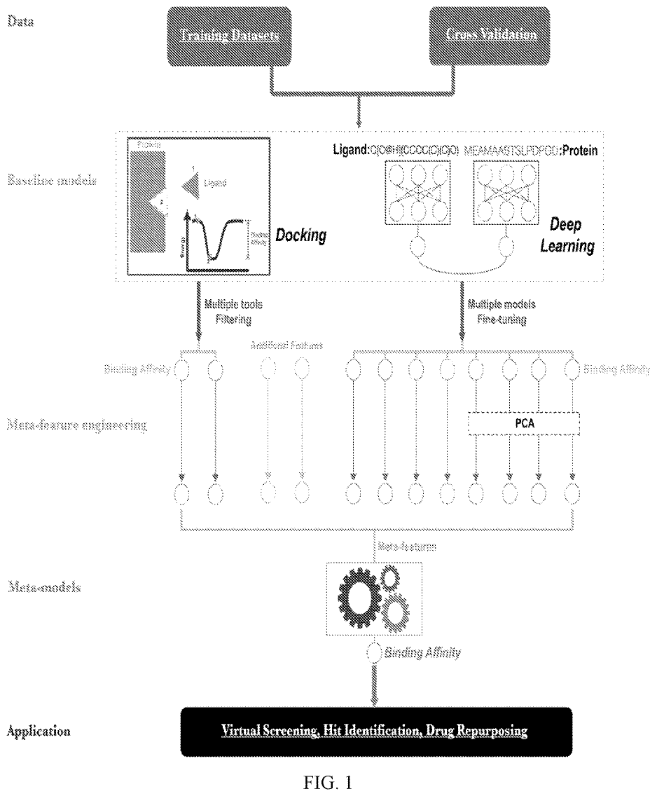

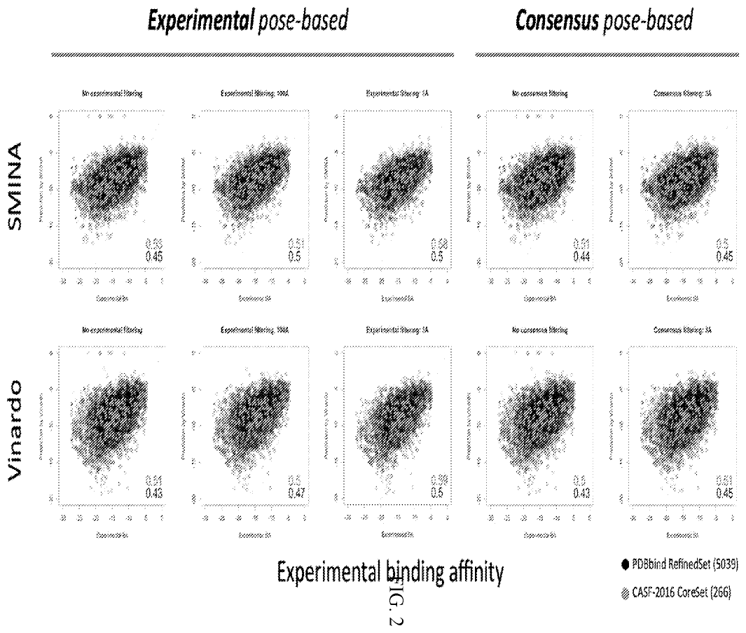

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±20% or ±10%, ±5%, ±1%, or ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

### Method of Training Algorithms

Provided herein are methods for predicting ligand-protein binding affinities. In some embodiments, the method includes training a machine learning model to predict ligand-protein binding affinity. In some embodiments, the machine learning model includes a machine learning meta-model. Meta-models, as used herein, refers to a type of ensemble learning by combining diverse algorithms, and ligand and protein features.

In some embodiments, training the machine learning model includes providing a meta-model training dataset, and training the machine learning meta-model on the training dataset to generate an algorithm for predicting ligand-protein binding affinities. The training dataset includes any suitable training data, such as, but not limited to, docking tool prediction data, deep learning (DL) prediction data, physiochemical properties (e.g., molecular weight), any other suitable data relating to the one or more ligand-protein complexes, or a combination thereof. In some embodiments, the training dataset includes at least two data types (e.g., two or more sets of docking tool prediction data; two or more sets of DL prediction data; at least one set of docking tool prediction data and at least one set of DL prediction data; at least one set of docking tool prediction data and physiochemical properties; at least one set of DL prediction data and physiochemical properties; at least one set of docking tool prediction data, at least one set of DL prediction data, and physiochemical properties). For example, in some embodiments, the method includes combining both classical scoring functions and deep learning (DL) models into a single integrative framework of meta-models (or fusion models).

In some embodiments, providing the meta-model training dataset includes generating at least one type of data for the meta-model training dataset. Generating at least one type of data may include generating at least one docking score for the binding affinity of one or more ligand protein complexes with one or more scoring functions and/or docking tools, generating at least one DL score for the one or more ligand-protein complexes with one or more DL models, or a combination thereof. For example, in some embodiments, the method includes generating at least one docking score for the binding affinity of one or more ligand-protein complexes using one or more structure-based docking tools and/or generating at least one DL score for the one or more ligand-protein complexes using one or more sequence-based DL models, generating the meta-model training dataset from the scores generated by at least two of docking, DL tools, and physicochemical properties, and training a machine learning meta-model on the training dataset to generate an algorithm for predicting ligand-protein binding affinities.

In some embodiments, generating the at least one docking score includes selecting a receptor structure file, generating docking poses and associated binding affinity scores for the docking poses for each of the one or more ligand-protein complexes by one or more docking tools, and analyzing the resulting docking poses for the one or more ligand-protein complexes. The analyzing step may include any suitable evaluation strategy, such as, but not limited to, consensus docking strategies based on either experimental or predicted ligand poses. Similarly, the docking score may be generated using any suitable scoring function, such as, but not limited to, an empirical scoring function (ESF). Suitable ESFs include, but are not limited to, SMINA and Vinardo (implemented in the computational docking program smina). In some embodiments, generating at least one docking score for the binding affinity comprises employing at least two ESFs in tandem. In such embodiments, the tandem use of at least two scoring functions (e.g., SMINA and Vinardo) addresses possible idiosyncrasies in the parameterizations of the scoring functions.

In some embodiments, the method further comprises preprocessing the docking scores for the one or more ligand poses in predicting each ligand-protein complex. The preprocessing may include filtering the docking scores through any suitable filtering strategy, such as, but not limited to, (a) filtering the docking poses output by empirical docking programs based on the degree to which they agree with experimental scores (experimental pose filtering), and/or (b) comparing poses from two or more docking methods, in so-called “consensus” approaches, and filtering based on the degree of agreement to yield a higher quality output (consensus pose filtering). For example, in some embodiments, the preprocessing comprises filtering the ligand poses based upon the experimental and/or predicted docking poses. In some embodiments, the filtering cut-off is about 3 Angstrom. In some embodiments, the filtering cut-off is 3 Angstrom. In some embodiments, the filtering cut-off is less than about 3 angstrom. In some embodiments, the filtering cut-off is 3 Angstroms (i.e. the RMSD should be <=3 Angstroms). In some embodiments, there is no filtering cut-off at all.

In some embodiments, the filtering comprises applying a root-mean-squared deviation (RMSD) filter. In some embodiments, the experimental pose filtering comprises calculating a deviation for at least one of the docking poses predicted by the ESF relative to an experimental structure of the ligand. In some embodiments, the consensus pose filtering comprises calculating a deviation between the docking poses predicted by one of the ESFs relative to the docking pose predicted by another one of the ESFs. Scores extracted from empirical docking runs of these parallel approaches to applying the RMSD filters are different when considering the experimental RMSD filter and the consensus RMSD filter. This is also the case when considering the unfiltered dataset, where the set of complexes are identical, but the choice of poses used to extract the best docking scores differ between the two RMSD calculation approaches.

In some embodiments, generating the at least one DL score comprises selecting at least one DL model to predict binding affinities of ligand-protein complexes, and generating scores for the one or more ligand-protein complexes using the at least one DL model. The DL model may include any suitable DL models. In some embodiments, the method includes multiple DL models of different architectures and/or multiple training strategies. Suitable DL models include, but are not limited to, CNN-CNN, Daylight-AAC, Daylight-CNN, Morgan-AAC, Morgan-CNN, MPNN-CNN, Transformer-CNN, MPNN-Quasi-seq, CNN/RNN-CNN/RNN, CNN-AAC, MPNN-Transformer, MPNN-PseudoAAC, MPNN-AAC, and/or MPNN-CNN/RNN. In some embodiments, the at least one DL model is a pre-trained model or a de novo-trained model. In some embodiments, the at least one pre-trained DL model includes CNN-CNN, Daylight-AAC, Morgan-AAC, Morgan-CNN, MPNN-CNN, and/or Transformer-CNN. In some embodiments, the at least one de novo-trained DL model includes CNN-AAC, MPNN-CNN, CNN-CNN, Daylight-CNN, MPNN-AAC, and/or MPNN-Transformer. In some embodiments, the at least one de novo-trained DL model includes CNN-AAC, MPNN-CNN, CNN-CNN, Daylight-CNN, MPNN-AAC, MPNN-Transformer, Daylight-AAC, Morgan-AAC, Morgan-CNN, and/or Transformer-CNN.

In some embodiments, selecting the at least one DL model comprises inputting ligand-encoding and receptor-encoding embeddings into one or more DL models, determining a performance of the one or more DL models, and selecting the at least one DL model based upon the performance of the one or more DL models. In some embodiments, the selecting comprises selecting the model that achieves the performances of mean-squared-error (MSE)<0.6. The ligand-encoding and the receptor-encoding embeddings may be from any suitable source, such as, but not limited to, DeepPurpose library. The DeepPurpose library offers a framework for various models that combine ligand-encoding and target-encoding neural networks.

Each DL model may be trained using any suitable strategy, library, and/or database. For example, in some embodiments, each DL model is either pre-trained or de novo-trained. The pre-trained models, when included, may be obtained from any suitable source, such as, but not limited to, an open-source DL library for drug-target interaction prediction (e.g., DeepPurpose). The de novo-trained models, when included, may be trained using any suitable database, such as, but not limited to, BindingDB, PDBbind, any other suitable database, or a combination thereof. In some embodiments, the pre-trained models and/or models trained with BindingDB are further fine-tuned using PDBbind data.

In some embodiments, one or more of the DL models are either de novo-trained or fine-tuned using 10 repetitions of 5-fold cross-validations (CVs). In some embodiments, principal component analysis (PCA) is performed on the DL scores from all CVs. In some embodiments, the prediction scores are generated from the first principal components of PCA. Additionally, or alternatively, in some embodiments, all prediction scores from repeated cross-validations for all DL models under study are merged to investigate top principal components from PCA as additional prediction scores.

In some embodiments, the method includes forming the meta-model from multiple machine learning models using the prediction scores from the docking tool (e.g., ESF) scores, DL model scores, and/or physiochemical properties. The meta-model may include any suitable number of machine learning models, such as, but not limited to, at least 2, at least 3, at least 4, at least 5, at least 6, at least 7,or any combination, sub-combination. For example, in some embodiments, the method includes taking all prediction scores from both ESF and DL models, as well as molecular weights of ligands, and building a meta-model from one of 4 machine learning algorithms, the meta-models forming the final tools for ligand-protein binding affinity prediction. In some embodiments, the meta-model is a linear model. In some embodiments, the meta-model is a non-linear model. The meta-model may be formed from any suitable machine learning model, such as, but not limited to, LASSO model, ElasticNet model, Linear Regression model, or XGBoost model.

While the docking tools according to one or more of the embodiments disclosed herein are based on scoring functions trained to match experimental structural data, the deep learning tools according to one or more of the embodiments disclosed herein capture information on the sequences of proteins and ligands. As such, the docking and deep learning tools together provide two independent approaches of quantifying the binding affinities, forming complementary sources of structure in the data. Thus, the meta-models according to one or more of the embodiments disclosed herein are able to leverage these two-independent approaches for predicting biding affinities of ligand-protein complexes. Such meta-models outperform individual base models and exhibit competitive performance to more sophisticated structure-based DL models.

### Apparatus

In another aspect, provided herein is an apparatus for predicting a ligand-protein binding affinity. In some embodiments, the apparatus comprises a processor, a memory unit; and a communication interface. In some embodiments, the processor is connected to the memory unit and the communication interface. In some embodiments, the processor and the memory are configured to implement the algorithm trained according to methods described elsewhere herein.

### A Computer Readable Storage Medium

In yet another aspect, provided herein is a non-transitory computer readable storage medium, storing computer-executable instructions for performing any of the method as described elsewhere herein.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in these experiments are now described.

### Methods

### Dataset Selection

### BindingDB

BindingDB ver. 2020m2 database (bindingdb.org) was used for the purpose of DL model training. A total of 66,444 ligand-protein complexes from the Ligand-Target-Affinity dataset were filtered with Kd<=0.01M, valid PubChem IDs or InChI keys for ligands, and valid UniProt IDs for proteins.

### PDBbind

The PDBbind v2020 refined set was downloaded from pdbbind.org.cn/. The dataset consisted of 5,316 target protein-drug ligand pairs. The refinedset has been filtered from a larger dataset by the removal of complexes that have “obvious problems in 3D structure, binding data or other aspects” (from the description in the Readme file “PDBbind-101.txt” included in the downloaded dataset). Of these, 285 ligand-protein complexes are in the so-called core set: these are high-quality data points that are used as the primary test set for the Comparative Assessment of Scoring Functions (CASF) benchmark. The core set was downloaded from the CASF-2016 link located at (www.pdbbind.org.cn/casf.php; Reference: Su M. Y. et al. Comparative Assessment of Scoring Functions: The CASF-2016 Update. J. Chem. Inf. Model., 2019. DOI: 10.1021/acs.jcim.8b00545). This core set was separated from the remainder of the PDBbind refined set to serve as the external test set (i.e., benchmark set) for evaluating machine learning model performance. Thus, before the application of further filters (described below), a training set of 5,031 ligand-protein complexes and an external test set of 285 complexes were considered. Binding affinity values used in this Example were the logarithms of the Kd or Ki values. The Kd and Ki values were treated as being identical for this analysis.

### Molecular Docking

### Implementation of Ligand Docking

The smina implementation (http://pubs.acs.org/doi/abs/10.1021/ci300604z) of the AutoDock Vina method was employed for the docking component. The smina implementation is designed to make AutoDock easier to use, with larger support for ligand molecular formats and improved minimization algorithms. Within this package, both the default SMINA scoring function, as well as an alternate scoring function, Vinardo (doi.org/10.1371/journal.pone.0155183), were used. [The italicized term smina describes the program, while the capitalized SMINA represents the scoring function.] In the meta-models, this class of empirical docking methods is referred to as E. The tandem use of both scoring functions is meant to address possible idiosyncrasies in the parameterizations of the scoring functions. smina is run as a command-line program using a receptor structure in PDBQT format and a ligand in 3D SDF format.

An in-house Python script includes steps for the preparation of the receptor structures, the creation of the configuration file, and the running of smina (described elsewhere herein). The output for each scoring function consists of the binding affinities for the 9 (the default option) best docking poses. Seven complexes with multiple ligands (whose names were separated with “&” or “/”) in the PDBbind dataset were not docked as there was some ambiguity as to which ligand to consider in the docking. These complexes were excluded from further analyses.

Custom Python wrapper scripts were constructed to implement the entire pipeline, with slight variations depending on the dataset considered. The primary aspects of running a docking simulation are as follows.
1. Selection of the receptor structure file: All data files for each complex in the PDBbind dataset are contained in separate subfolders. The protein PDB file was utilized for the remaining processing steps.
2. Preparation of the receptor structure: The receptor structure is read in as a PDB file and preprocessed in PyMol (pymol.org). The pymol class and associated functions were used within the pipeline script. The preprocessing consists of the removal of all solvent and heteroatoms in the molecules. The resulting PDB file is then converted into the requisite PDBQT format using OpenBabel (openbabel.org). The obabel flags ‘-e=continue after errors’, ‘-p=add hydrogens appropriate for the pH’, and ‘--partialcharge gasteiger’ were used to calculate Gasteiger partial charges. The pH was set at 7.0. We included the PDBQT-specific flags ‘r=output as a rigid molecular’ and ‘p=Preserve atom indices from input file’. The implementation of this is written as
   obabel -ipdb <Input PDB file> -opdbqt -O <Output PDBQT file> -p 7.0 -e -xrp --partialcharge gasteiger
(Note the ‘-x’ flag is set to enable the output-specific flags, which are PDBQT-specific in this case.).
3. Ligand files: Given the use of 3D SDF files for all analyses, the ligand files did not need further processing. The appropriate ligand SDF files were invoked in the docking command (see below), including the complex-specific ligand files in the PDBbind dataset (the ligand SDF files are included in the subfolders for each complex).
4. Creation of the configuration file: The configuration file serves to provide a set of commands to the smina package (as well as the original AutoDock tool). The file contains the receptor PDBQT file, the ligand sdf file, the center coordinates of the search box in the receptor structure file coordinate system, and the dimensions of the search box. The calculation of the center coordinates varies depending on the datasets under consideration. For the PDBbind dataset, the center of mass of each ligand SDF file was used as the center of the search box, employing PyMol's centerofmass function to do so. For the 3CL protease dataset, we used the center of mass of the ligand (O6K) found in the 6y2f.pdb file as the center of the search box. The search box was set to have dimensions of 40 Angstroms in the x, y and z directions.
5. Running the docking software: Using the configuration file and the ligand file name, the program smina was ran using both the default (hereafter termed “SMINA”) and “Vinardo” scoring functions. An exhaustiveness parameter of 32, and the default output of the top 9 poses (ranked from 0 to 8 from lowest to highest binding energy) were used. The program call was:
   smina --config <config_file> --out <Output Complex PDBQT file> --log <Log file containing binding affinities> --exhaustiveness 32
Seven complexes with multiple ligands (whose names were separated with an “&” or “/”) were not docked as there was some ambiguity as to which ligand to consider in the docking. These complexes were not included in further analyses.

### Processing of Docking Scores

Previous studies have demonstrated the value of (a) filtering the docking poses output by empirical docking programs based on the degree to which they agree with experimental scores (Koes, D. R. et. al. J Chem Inf Model 2013, 53 (8), 1893-904) and (b) comparing poses from two or more docking methods, in so-called “consensus” approaches, and filtering based on the degree of agreement to yield a higher quality output (Houston, D. R. et al., Journal of Chemical Information and Modeling 2013, 53 (2), 384-390; Palacio-Rodriguez, K et al. Scientific reports 2019, 9 (1), 5142). Accordingly, preprocessing of the docking scores was carried out by filtering out certain complexes based on the quality of the associated poses assessed by these two criteria. For each complex out of the available 9 docking poses, root-mean-square deviation (RMSD) was calculated between docking poses in two different ways, and the RMSD value was utilized as a cutoff in determining the best set of binding affinities to choose in the final dataset to the meta-models. The goal was to determine if pose quality, either relative to an available experimental gold-standard or relative to the results of another scoring function, would impact both the matching of the predicted binding affinity as well as the downstream prediction process.

It was chosen to implement the symmetric RMSD employed in Trott and Olson 2010 (Journal of Computational Chemistry (2010), 31 (2), 455-461), using Pymol's Python API. In brief, this RMSD is calculated between two different ligand structures as RMSD_{ab}=max(R_{a|b}, R_{b|a}) where R_{a|b} describes an asymmetric measure of distance:

$${R}_{a \vert b} = \sqrt{\frac{1}{N} {\sum }_{i} {min}_{j} {r}_{ij}^{2}}$$

where the summation is over all N heavy atoms in structure a, the minimum is over all atoms (j) in structure b of the same element type as atom i in structure a.

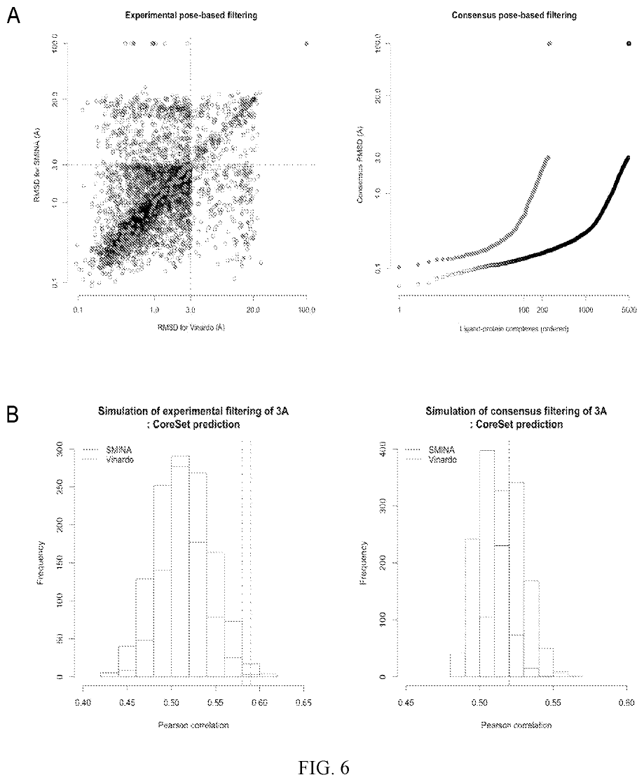

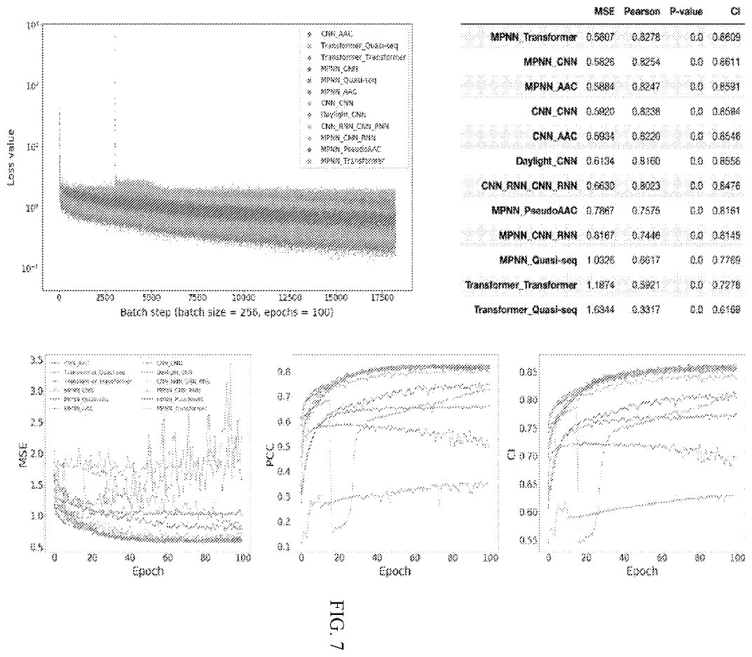

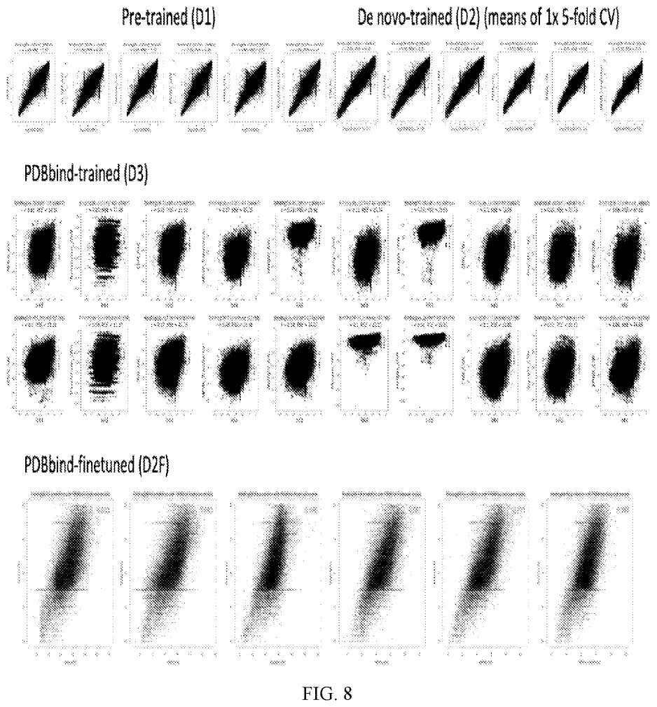

RMSD_{ab} was calculated in two ways, depending on the choice of docking poses: (1) Experimental pose-RMSD filtering, where the RMSD is calculated for a ligand pose predicted by a docking method relative to the experimental structure of the ligand (when available); and (2) Consensus pose-RMSD filtering, where the RMSD is calculated between the docking poses for the same ligand-target pair as predicted by two different docking scoring methods, Vinardo and SMINA. Details of these filtering approaches are as follows:
a. Experimental pose-RMSD filtering: The deviation is calculated for a ligand pose predicted by a docking method relative to the experimental structure of the ligand. The experimental structures are available, in the PDBbind dataset in sdf and mol2 formats. The calculation of this RMSD in Pymol's Python API involved first reordering the bonds in the docking pose using either the experimental structure or the SMILES string as a “template”, followed by the calculation of the RMSD between the experimental and reordered predicted pose. Ligands were assigned an RMSD of 100 Angstroms if either the docking tools failed to generate a docked structure, or if the reordering process failed. In those cases, the lowest energy pose out of the 9 output was automatically selected by the docking tools. The bond reordering process was carried out using the experimental mol2 file (for the PDBbind structures), using functions in the Python package RDKit (www.rdkit.org/). After reordering, the symmetric RMSD was calculated as described above, and the poses were filtered for each complex by an RMSD cutoff of 3 Angstroms. Among those poses that satisfied the cutoff the lowest energy structure was chosen. If no poses met the cutoff, then the lowest energy pose with the RMSD set to 100 Angstroms was selected. In this way, two tiers of poses were there from all the complexes: a set of docked poses with RMSD relative to the experimental poses <3 Angstroms (and which may not have been the lowest energy pose among the 9 docking poses); and another set where the lowest energy pose was selected with an arbitrarily set RMSD of 100 Angstroms (for downstream filtering purposes).
b. Consensus pose-RMSD filtering: This is where the deviation is calculated between the docking poses for the same ligand-target pair as predicted by two different docking methods. In this analyses, these correspond to the poses from the SMINA and Vinardo scoring functions. For the Vinardo and SMINA pose comparisons, 9×9=81 pairs of docked poses were there to filter through for the final assignment of RMSD. In the case of docked pose comparisons, there was no need for bond reordering, as the smina program generated poses with the same ordering convention using both scoring functions. The RMSD was calculated for all pairs of docking poses for each complex and the [SMINA, Vinardo] pair with an RMSD less than 3 Angstroms and with the lowest rank sum (SMINA rank+Vinardo rank) was chosen in the final scoring. If multiple pairs had the same rank sum, the pair with the lowest RMSD was selected. If a complex had no pairs with an RMSD less than 3 Angstroms, then the lowest energy poses from both methods were selected and assigned a pairwise RMSD of 100 Angstroms for downstream filtering. As a result of this assignment process, all structures with an RMSD<100 Angstroms will necessarily have an RMSD<3 Angstroms. Thus, it was needed to consider only one of the two cases in the results.
   A cutoff of 3 Angstroms was chosen as representing a reasonably good quality match between poses based on manual inspection of RMSD distributions (FIG. 6). This is a more lenient cutoff relative to other analyses (e.g. doi.org/10.1021/ci300604z, dx.doi.org/10.1021/ci300399w) that suggest a 2-Angstrom cutoff. This approach is a compromise between the need for higher quality matches, and the need to have sufficient numbers of data points to train the models as stricter cutoffs lead to smaller dataset sizes. Note on the coding of Experimental pose-RMSD filtering: The experimental structures are available, in analyses, in the PDBbind dataset in SDF and MOL2 formats. The calculation of this RMSD in Pymol's Python API (www.pymol.org/pymol) involved first reordering the bonds in the docking pose using either the experimental structure or the SMILES string as a “template”, as the atoms are not consistently designated between the smina predicted poses and the experimental structures. This was followed by the calculation of the RMSD between the experimental and reordered predicted pose. The bond reordering process was carried out using the experimental mol2 file (for the PDBbind structures), using functions in the Python package RDKit (www.rdkit.org/).

An important aspect of these parallel approaches to applying the RMSD filters on the data is that the scores extracted from the empirical docking runs are different when considering the Experimental RMSD filter and the Consensus RMSD filter. This is even the case when considering the unfiltered dataset, where the set of complexes are identical, but the choice of poses used to extract the best docking scores differ between the two RMSD calculation approaches.

### Deep Learning

### DeepPurpose Library

Python library, DeepPurpose, was used for deep learning of binding affinities of ligand-target complexes. It offers a framework for various models that combine ligand-encoding and target-encoding neural networks. The version 0.0.1 of the library (as of Jul. 24, 2020) was modified and customized for development of this work, while all 6 pre-trained models with BindingDB-2020m2-Kd were downloaded from the version 0.1.5 (as of Jun. 29, 2022). The 6 pre-trained models are CNN-CNN, Daylight-AAC, Morgan-AAC, Morgan-CNN, MPNN-CNN, and Transformer-CNN. This class of BindingDB pre-trained models is referred to as D1

Training of 12 DL Models from the DeepPurpose Library Using BindingDB

The DeepPurpose library offers various embedding methods for both ligands and proteins to build regression models to predict binding affinities of ligand-protein complexes. After initial pilot experiments, the following 12 models of ligand-protein embedding methods were chosen for downstream analyses: CNN-CNN, Daylight-AAC, Morgan-AAC, Morgan-CNN, MPNN-CNN, Transformer-CNN, MPNN-Quasi-seq, CNN/RNN-CNN/RNN, CNN-AAC, MPNN-Transformer, MPNN-PseudoAAC, MPNN-AAC, MPNN-CNN/RNN, Transformer-Quasi-seq, and Transformer-Transformer. These 12 models were applied to BindingDB Kd data, which were used by the DeepPurpose library. Their 6 pre-trained models mentioned above achieved performances of MSE<0.6 or Pearson correlation coefficient (PCC)<0.8. Given the published results of their best models, all 12 models were re-trained or de novo-trained from scratch to select models with similar performances for downstream model development. All default were used hyperparameters for each model in DeepPurpose and did not perform cross validation given the target performance of MSE=0.6 or PCC=0.8. The data split for training, validation, and testing was 70% (46,511 cpx), 10% (6,644 cpx), and 20% (13,289 cpx), respectively, with random seed=1. Those top models selected with the target performance are considered as crude or low-confidence deep learning models, being a starting point in the entire pipeline for the sake of computational efficiency given limited resources. This class of BindingDB de novo-trained models are referred to as D2

### Training of DL Models Using PDBbind

10 DL models were also trained using the PDBbind RefinedSet binding affinity data (Kd/Ki) that contain ligand-target structural data and were used by the docking tools. The 10 models come from the 6 pre-trained models and the top 6 de novo-trained models selected from above (two models in common). This is to compare with the BindingDB-trained DL models. The CASF2016 CoreSet within the PDBbind RefinedSet, which is used for external validation was excluded, and 10× repeated 5-fold cross validation with random seed=1701 was performed. This class of PDBbind-trained models are referred to as D3

### Fine-Tuning of the BDB-Trained Models Using PDBbind

In order to leverage the respectable performance of the BindingDB-trained models (D1 and D2), they were finetuned using the PDBbind data excluding the CoreSet (referred as D1F and D2F, respectively). To increase analysis robustness, iterative 5-fold CV was performed with 10 iterations, i.e., 50 validations (using the scikit-learn function in Python, RepeatedKFold or KFold, with random_state=1701) for each of the 6 D1F, 6 D2F, and 10 D3 models to yield 300, 300, 500 model variants or predictions, respectively. PCA was also performed on those predictions for the 3 model classes (referred as D1FP, D2FP, D3P, respectively) as well as on the merged 1,100 predictions (referred as DAP) for ML meta-models below for dimensionality reduction of the meta-features (predictors).

### Machine Learning Meta-Models

The fact that there are two independent means of quantifying the binding affinities (i.e. docking and deep learning tools) means that the data potentially comprises complementary sources of structure. The docking tools are based on scoring functions trained to match experimental structural data, while the deep learning tools capture information on the sequences of proteins and ligands. The exact partition of dual sequence/structure impacts on the results from these two approaches is not entirely clear, but the expectation is that there is likely non-overlapping information contained in the predictions of these approaches. Accordingly, it was sought to exploit any complementary information by feeding the docking and deep learning scores into a set of linear and non-linear machine learning models to gain improvements in prediction of binding affinities. These machine learning models are termed as meta-models since they incorporate predictions from independent tools as base models. Linear regression, ElasticNet, and LASSO algorithms were used for linear meta-models and the XGBoost algorithm was used for a non-linear meta-model implemented in scikit-learn in Python. Hyperparameter optimization was done along with multiple 5-fold cross validations.

The steps in the preprocessing of the PDBbind dataset were as follows:
1. The predicted binding affinity matrices from the docking and deep learning tools were merged based on the PDB IDs.
2. The RMSD values were separately incorporated for the complexes (either based on ‘Experimental’ or ‘Consensus’ quantifications) into the merged binding affinity matrix.
3. Those structures in the non-core-set not satisfying the RMSD cutoffs were removed from further analysis. The RMSD filters were not applied to the core set.
4. The molecular weight of the ligands were also calculated and the possibility of incorporating these values into the binding affinity matrix (this is enabled by a separate flag in the code, which either includes or excludes the molecular weight) was allowed.
5. All structures in the core set of the PDBbind cohort are set aside for the test scoring. The remainder of the binding affinity matrix is carried forward for the analysis.
6. The binding affinity matrix for the training set (the non-core-set portion of the PDBbind refined set) was separated into feature (X) and target value (y) numpy arrays.

These training sets were input to the meta-models. The parameters used in each of the models are:
A. LASSO model: scikit-learn's LassoCV approach was iterated 100 times with shuffled data, using 5-fold cross-validation each time. The best model chosen was the one with the minimum ‘dual_gap_’ score.
B. ElasticNet model: scikit-learn's ElasticNetCV approach was iterated 10 times for each L1_ratio in the list [0.1, 0.5, 0.7, 0.9, 0.95, 0.99, 1] (the L1_ratio parameter is the balance between the L1 and L2 penalties in the ElasticNet method). Each iteration used shuffled data and 5-fold cross-validation. The best model chosen overall was the one with the minimum ‘dual_gap_’ score.
C. Linear Regression model: linear regression with no additional parameters or regularization penalties was run.
D. XGBoost model (https://xgboost.readthedocs.io/en/release_0.72/python/): The xgboost.XGBRegressor model class with a “squared-error” objective was used. This model was then used in scikit-learn's RandomizedSearchCV method to run 100 iterations with 5-fold cross-validation, comprehensively exploring several parameters within reasonable ranges: “colsample_bytree” uniformly in the range [0.8, 0.2]; “gamma” uniformly in the range [0, 0.5]; “learning_rate” uniformly in the range [0.02, 0.3]; “max_depth” randomly from the set of integers in the range [2, 6], “n_estimators” randomly from the set of integers in the range [100, 150]; “subsample” uniformly in the range [0.7, 0.3]. The maximum ‘mean_test_score’ was used to select the best model.

The features for the meta-models are SMINA and Vinardo predictions with different RMSD filtering cutoffs, ligand molecular weights, and DL predictions. Various combinations of those features were experimented with: (1) SMINA+Vinardo (with/without the filters) (2) SMINA+Vinardo (with/without the filters)+MW (3) SMINA+Vinardo+MW+DL mean predictions (BindingDB or PDBbind) and (4) SMINA+Vinardo+MW+DL PCA projections (BindingDB or PDBbind). For the BindingDB-trained scores, the top 6 models were used as described above. For the DL models fine-tuned on the PDBbind dataset, the mean of the 50 CV predictions was taken for each of the 6 models (i.e., 6 meta-model features). For the PCA of all DL scores, an optimization was ran over the number of principal components (PCs) to include as features. Between 1 and 22 top principal components were allowed to be included (i.e., PC 1, PCs 1-2, PCs 1-3, . . . PCs 1-22), and the selection of the best model among the 22 options was done by maximizing the Pearson's correlation for each of the 4 meta-models separately using 20% of the training data as a leave-out validation set for hyperparameter optimization. It was chosen to optimize up to the top 22 PCs to match the number of models used in all the deep learning runs in D1, D2 and D3 combined.

Each of the optimized meta-models generated above is tested against the core set using the relevant meta-model-specific feature sets. For all meta-models, the Pearson's correlation, the Spearman rank correlation, the mean-square error (MSE) and the root-mean-square error (RMSE) for the core-set predictions were calculated and reported for model evaluation. A detailed description of these meta-model features is shown in Table 1.

TABLE 1
Descriptions of meta-model feature groups

| Feature | | |
|---|---|---|
| Group ID | Description of features included | Feature list |
| E | Smina and Vinardo empirical (E) docking scores | Smina score, Vinardio score |
| | only | |
| EW | E + molecular weight (MW) | Smina score, Vinardo score, MW |
| ED1 | E + MW + Deep Learning (D) average scores from | Smina score, Vinardo score, MW, CNN_CNN, |
| | 6 BindingDB- pre-trained models provided in | Day light_AAC, Morgan_AAC, Morgan_CNN, |
| | DeepPurpose v0.1.5 | MPNN_CNN, Transformer_CNN |
| ED2 | E + MW + D average scores from 6 models from | Smina score, Vinardo score, MW, CNN_AAC, |
| | DeepPurpose v0.0.1 trained de novo on BindingDB | MPNN_CNN, CNN_CNN, Day light_CNN, |
| | | MPNN_AAC, MPNN_Transformer |
| ED3 | E + MW + D average scores from 10 models from | Smina score, Vinardo score, MW, CNN_AAC, |
| | DeepPurpose 0.0.1 trained de novo on PDBbind | MPNN_CNN, CNN_CNN, Day light_CNN, |
| | | MPNN_AAC, MPNN_Transformer, Daylight_AAC, |
| | | Morgan_AAC, Morgan_CNN, Transformer_CNN |
| ED1-F | E + MW + D average of scores obtained from fine- | Smina score, Vinardo score, MW, CNN_CNN, |
| | tuning the ED1 models, using PDBBind v 2020 (the | Day light_AAC, Morgan_AAC, Morgan_CNN, |
| | D scores are averaged for each model across 5 | MPNN_CNN, Transformer_CNN |
| | folds of cross-validation X 10 repetitions) | |
| ED2-F | E + MW + D average of scores obtained from fine- | Smina score, Vinardo score, MW, CNN_AAC, |
| | tuning the ED2 models, using PDBBind v 2020 (the | MPNN_CNN, CNN_CNN, Day light_CNN, |
| | D scores are averaged for each model across 5 | MPNN_AAC, MPNN_Transformer |
| | folds of cross-validation X 10 repetitions) | |
| ED1-F-P | E + MW + D, where the reported scores are | Smina score, Vinardo score, MW, Up to 22 top PCs |
| | obtained by applying PCA to all scores from the 5 | (determined for each machine learning method |
| | folds of cross-validation X 10 repetitions for the six | through hyperparameter optimization) |
| | models in ED1-F | |
| ED2-F-P | E + MW + D, where the reported scores are | Smina score, Vinerdo score, MW, Up to 22 top PCs |
| | obtained by applying PCA to all scores from the 5 | (determined for each machine learning method |
| | folds of cross-validation X 10 repetitions for the six | through hyperparameter optimization) |
| | models in ED2-F | |
| ED3-P | E + MW +D, where the reported scores are | Smina score, Vinardo score, MW, Up to 22 top PCs |
| | obtained by applying PCA to all scores from the 5 | (determined for each machine learning method |
| | folds of cross-validation X 10 repetitions for the six | through hyperparameter optimization) |
| | models in ED3 | |
| ED-A-P | E + MW + D, where the reported scores are | Smina score, Vinardo score, MW, Up to 22 top PCs |
| | obtained by applying PCA to all scores from the 5 | (determined for each machine learning method |
| | folds of cross-validation X 10 repetitions for the | through hyperparameter optimization) |
| | models in ED1-F, ED2-F and ED3 | |

### SwissADME Analysis

To understand the contributions of ligand properties to performance of all the models, the web tool SwissADME (nature.com/articles/srep42717) was used to extract the properties of the PDBbind CoreSet ligands from their SMILES strings. The ligand SMILES strings were first converted to canonical SMILES format using OpenBabel (openbabel.org). The canonical SMILEs strings were then submitted to SwissADME's web interface. This yielded a set of 46 properties for 263 ligands in the CoreSet. Subsequently it was sought to measure the correlation between the deviation of the predicted scores from the experimental values with the numerical features, to identify those ligand features that might contribute to reduced performance of the model.

The SwissADME features that might be associated with better or worse performance for subsets of complexes by the different groups of tools (docking, DL, and meta-models) were also investigated. Duplicate SMILES strings for the ligands were checked for and none were found, thus requiring no further filtration. Using two-sided Wilcoxon and t-tests, the distributions for all the numeric SwissADME features were compared.

### UniProt Analysis

To identify protein features that might be associated with better or worse performance of specific tools, the relevant subsets of PDBbind CoreSet PDB IDs were extracted from the analyses and fed into the “Retrieve/ID mapping” tool of the UniProt database (uniprot.org/id-mapping). The corresponding UniProt IDs and the following features were downloaded in a .tsv file: ‘From’, ‘Entry’, ‘Reviewed’, ‘Entry Name’, ‘Protein names’, ‘Gene Names’, ‘Organism’, ‘Length’, ‘Active site’, ‘Binding site’, ‘Function [CC]’, ‘Catalytic activity’, ‘Annotation’, ‘Gene Ontology (biological process)’, ‘Helix’, ‘Turn’, ‘Beta strand’, ‘Coiled coil’, ‘Compositional bias’, ‘Domain [CC]’, ‘Domain [FT]’, ‘Motif’, ‘Protein families’, ‘Region’, ‘Repeat’, ‘Zinc finger’, ‘Pathway’, ‘Gene Ontology (molecular function)’, ‘Post-translational modification’. All UniProt entries that were not labeled “reviewed” were first filtered out. Next, repeats were manually checked, where the same PDB ID mapped to multiple UniProt IDs, by looking up the PDB IDs on the RCSB PDB website (www.rcsb.org/). The “Gene Names” and “Species” were checked and all UniProt entries that did not match the PDB entry were removed. In all cases, this left only a single PDB ID-UniProt ID mapping for each PDB ID. Additionally, to run the tests of over- or under-representation, the UniProt IDs were also deduplicated. That is, all cases of proteins that were represented more than once were removed, as the CoreSet has a large number of proteins that recur in the dataset. This led to a significant reduction in the number of proteins tested for UniProt feature prioritization from 266 to 60. Fisher's exact test was run when comparing the number of occurrences of a feature in the subset versus the whole. The features tested are: the number of times a given UniProt term was annotated at all; the occurrence of Gene Ontology 7 terms, both the “Biological Process” and “Molecular Function” categories; and protein Domain and Pathway annotations.

### Virtual Screening Benchmark

To test the possibility of certain tools performing the prediction task with differing degrees of accuracy for certain proteins or even families of proteins, a virtual screening test by was devised by selecting 3 most frequently targeted proteins by multiple ligands from the general set of PDBbind v2020 (GeneralSet) excluding the RefinedSet used for model training. Only those proteins in GeneralSet were considered whose sequence similarity to those in RefinedSet was found to be less than 30% by the NCBI Blastp package. Complexes in the GeneralSet that had only IC50 experimental measurements were also filtered out for consistency with this study. The resulting top 3 proteins are O60341, O15151, and P0C6U8 (UniProt Accession IDs), which are complexed with 20, 8, and 7 ligands, respectively. The resolution of the 35 complexes ranges from 1.33 to 3.50 Angstroms with mean of 2.58 and standard deviation of 0.69.

### Example 1: Docking Tools

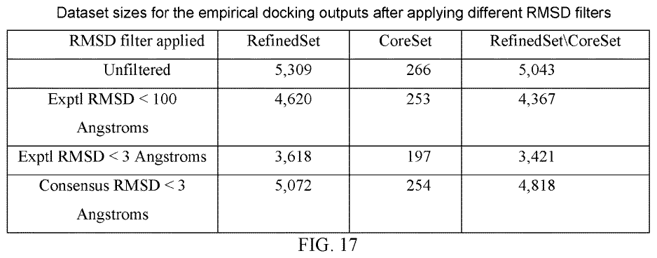

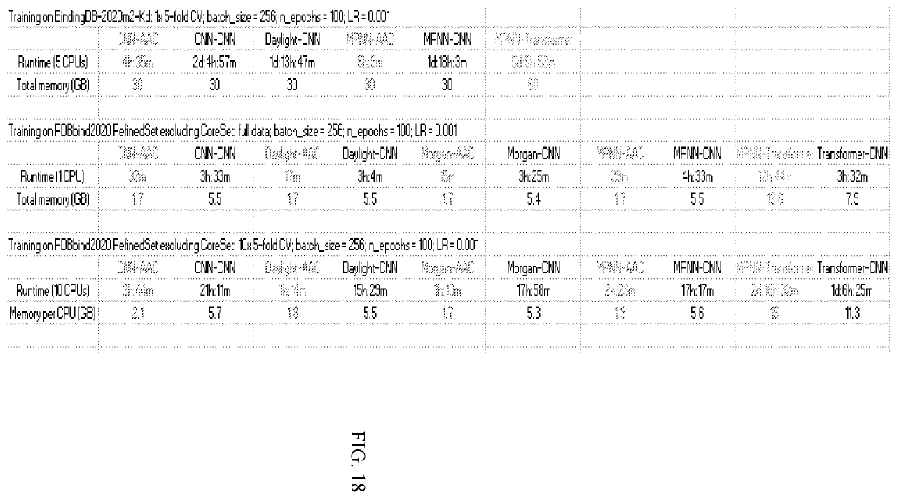

smina was run with two different scoring functions, SMINA and Vinardo, on the PDBbind dataset. The PDBbind “RefinedSet” excluding the “CoreSet” (designated as “RefinedSetCoreSet”) was distinguished from the “CoreSet” which is used as the external validation (benchmark) set in this Example. Additionally, as described in the methods, two formulations of the docking pose-based RMSD were considered for the 9 poses generated by the docking tools for each protein-ligand complex: an “Experimental” RMSD relative to the experimental pose, and a “Consensus” RMSD. The unfiltered dataset was indicated by RMSD<101 Angstroms, as all structures satisfy this condition based on the set-up. SMINA and Vinardo scores were compared against the experimental binding affinities using both RMSD filter types for the two scoring functions and cutoffs of 101 Angstroms, 100 Angstroms and 3 Angstroms (FIG. 2). The sizes of the RefinedSetCoreSet satisfying the filters are shown in FIG. 17.

The results indicate that the outputs of the docking tools consistently have a moderate correlation with the experimental results (on the order of 0.5), for both the RefinedSetCoreSet, and the CoreSet. Upon restricting the Experimental RMSD cutoff, the Pearson's correlation with the experimental docking scores (both SMINA and Vinardo) improves to 0.58-0.59 for the RefinedSetCoreSet at RMSD<100 Angstroms, while the correlation improves for the CoreSet only for RMSD<3 Angstroms. The latter effect could be a result of either a legitimate increase in prediction quality when restricting the Experimental RMSD for the CoreSet, or could simply be the stochastic impact of a reduction in the dataset size. To test this, Monte Carlo simulations were run, randomly subsetting 197 out of 266 complexes a thousand times and calculating the resulting correlation with experimental affinities. The empirical null distributions demonstrate that the improvement is unlikely to be a statistical artifact of subsampling (FIG. 6). Overall, this indicates that constraining the pose to be similar to the experimental pose has benefits for the quality of the docking score prediction. The results for the Consensus RMSD do not suggest any significant improvement by lowering the RMSD cutoff (only a slight improvement is observed for the RefinedSetCoreSet).

Additionally, it is interesting to note that the prediction on the CoreSet is consistently better than that of the RefinedSetCoreSet. The most obvious explanation for this is that the scoring function for Vinardo (doi.org/10.1371/journal.pone.0155183) was trained on the PDBbind 2013 CoreSet. This dataset overlaps with the PDBbind 2016 CoreSet used herein. The SMINA scoring function was trained on the CSAR-NRC HiQ 2010 data set ([PubMed Central: PMC3180202] DOI: 10.1021/ci200082t), which includes some structures from PDBbind RefinedSet from 2007. These overlaps will result in better performance on the CoreSet. Another possibility is that the complexes in the CoreSet are, by design, of a higher quality in terms of the removal of steric clashes and other structural irregularities relative to the RefinedSetCoreSet. Working with such protein-ligand pairs may result in a better fit to the experimental binding affinities. It was also noted that the SMINA and Vinardo scores are highly correlated with each other (FIG. 6), a fact that impacts meta-modeling later, as two strongly collinear features compete with each other when input into the same model.

It was concluded that the overall performances of the docking results are not strong enough to make reliable predictions. The results motivated exploring other alternative methods of binding affinity prediction or scoring, especially deep learning tools.

### Example 2: Deep Learning Models

Trained models using BindingDB or PDBbind-RefinedSet

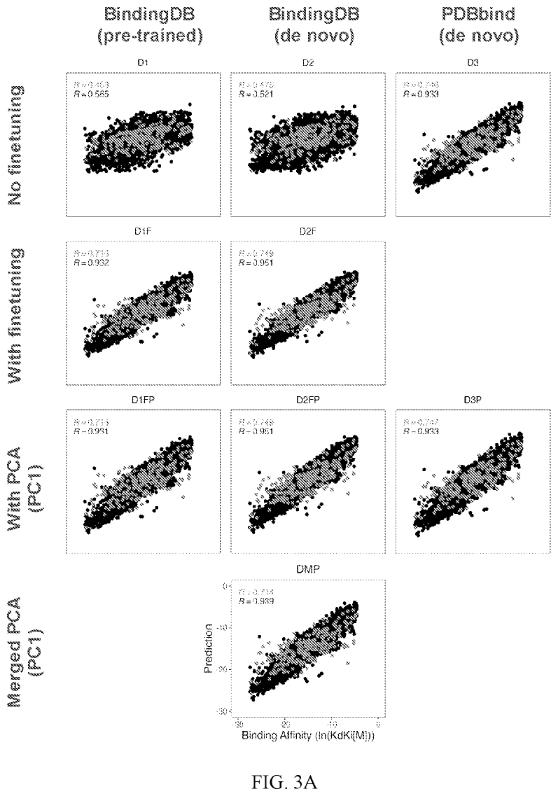

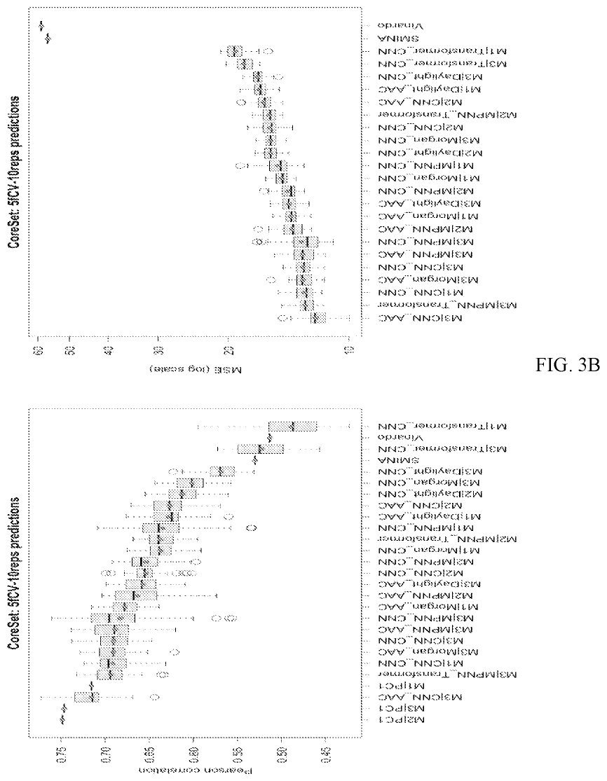

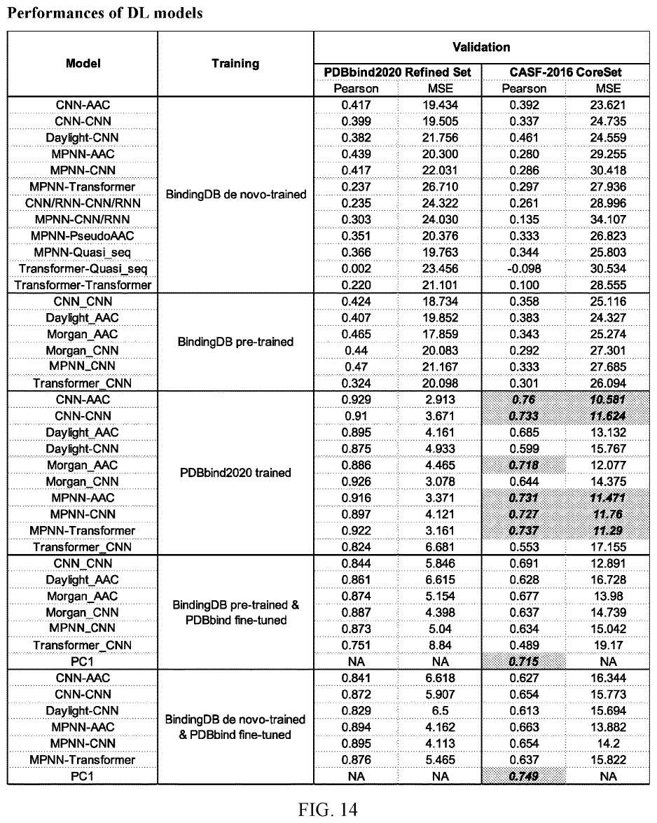

As the first group of DL models, either pre-trained (i.e., published) or de novo-trained models were investigated using the BindingDB (BDB) or PDBbind-RefinedSet (PDBb) data (model class D1, D2, or D3, respectively) (Table shown in FIG. 14). Based on mean-squared-error (MSE), the following top 6 out of the 12 de novo-trained models were selected for subsequent analyses, which show comparable performance to the DeepPurpose pre-trained models with MSE<0.6: CNN-CNN, Daylight-AAC, Morgan-AAC, Morgan-CNN, MPNN-CNN, and Transformer-CNN (FIG. 7). As the overlap between the published and de novo-trained models is only two, CNN-CNN and MPNN-CNN, this expanded the family of BDB-trained base models to a total of 10 with competitive performances in this study (FIG. 14). In terms of mean cross-validation (from 10×5-fold CV), the best performing BDB-pre-trained, BDB-de-novo-trained, and PDBb-trained models were Daylight-AAC, Daylight-CNN, and CNN-AAC (FIG. 14). The model average Pearson correlation coefficients were 0.468, 0.475, and 0.746 for the CoreSet, respectively (FIG. 3A; D1, D2, and D3). The two BDB-trained models perform worse than the docking tools (FIG. 2). Full performance results are provided in FIG. 18. It is noted that the PDBb-trained models performed poorly for BDB-Kd predictions with no correlations (FIG. 8).

### Fine-Tuned Models Using PDBbind-RefinedSet

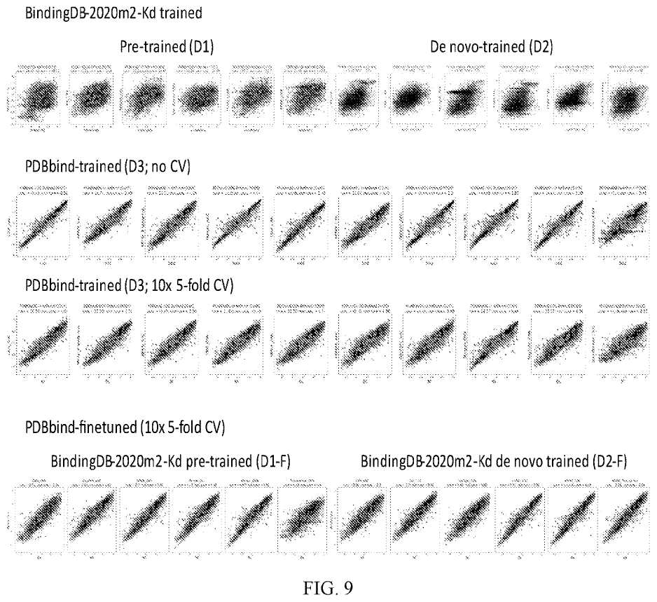

As the second group of models, fine-tuned models using PDBb were investigated for both BDB-pre-trained and BDB-de-novo-trained models (D1F and D2F, respectively) in the first model group above. In terms of mean cross-validation (from 10×5-fold CV), the best performing PDBb-finetuned models for BDB-pre-trained and BDB-de-novo-trained models were CNN-CNN and MPNN-AAC with Pearson correlation coefficients of 0.716 and 0.749 for the CoreSet, respectively (FIG. 3A, second row; FIG. 3B). Full performance results were obtained (Data not shown). As for BDB-Kd prediction, all PDBb-finetuned models maintained good performances with Pearson correlations of 0.6-0.7, compared to predictions by the BDB-trained models (FIGS. 8 and 9).

### PCA of Predictions

As the third or final group of DL models, PCA projections of all predictions from those fine-tuned models for the BDB-pre-trained (D1) or BDB-de-novo-trained (D2) in the second model group (6*5*10=300 D1 or D2 model instances from 10×5-fold CV for each model architecture) or those PDBb-trained models (D3) in the first model group (10*5*10=500 model instances) were investigated. The first principal component (PC1) shows the best performance in all PCA, Pearson correlation coefficients for D1, D2, and D3 being 0.715, 0.749, and 0.747, respectively (FIG. 3A, third row; FIG. 3B). PCA was also performed on the merged set of all predictions from D1, D2, and D3 (300+300+500=1100 model instances), Pearson correlation coefficient with PC1 being 0.744 (FIG. 3A, bottom). As expected, the PC1 is highly correlated with the mean prediction with the largest Pearson correlation coefficient of >0.99 in all cases, while the second most correlated PC was not the second PC (PC2). By looking at performances of all cross-validated fine-tuned or PCA models, it was also observed that PC1 and D3 models tend to perform better than D1 or D2 models for the CoreSet in terms of Pearson correlation coefficients or mean squared errors (FIG. 3B).

### Comparisons Among DL and Docking Models

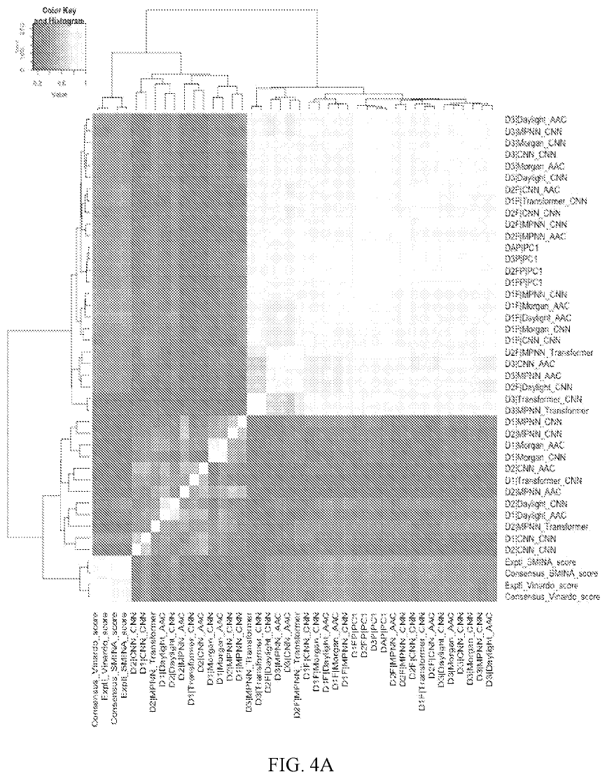

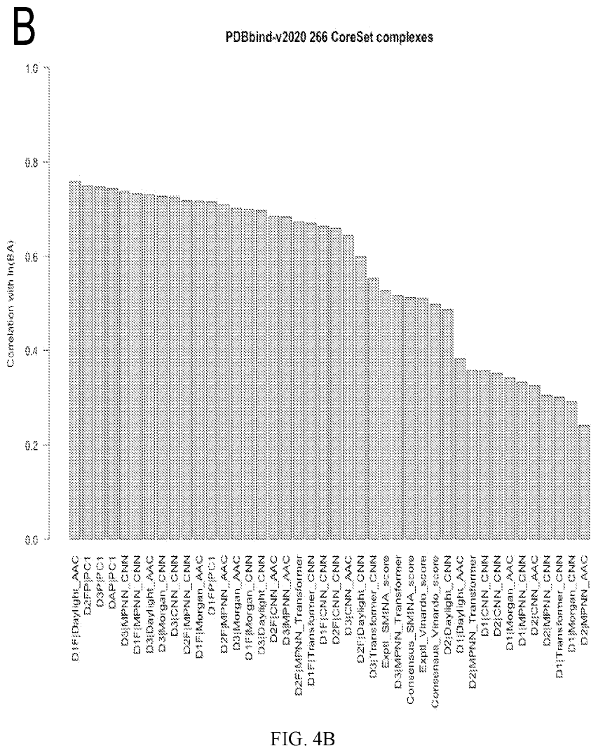

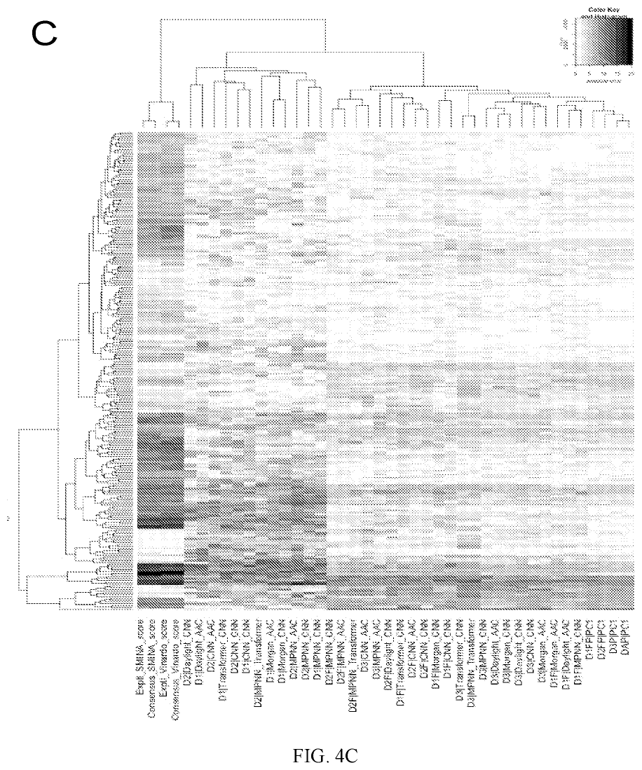

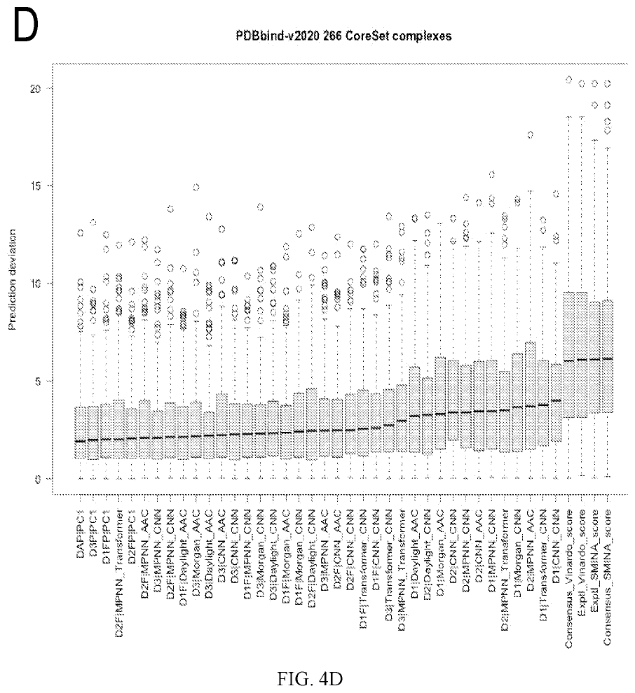

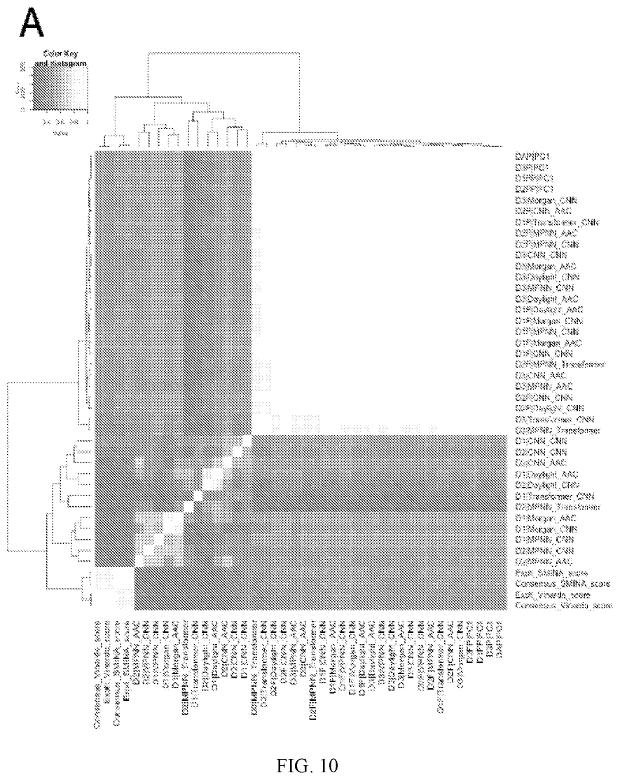

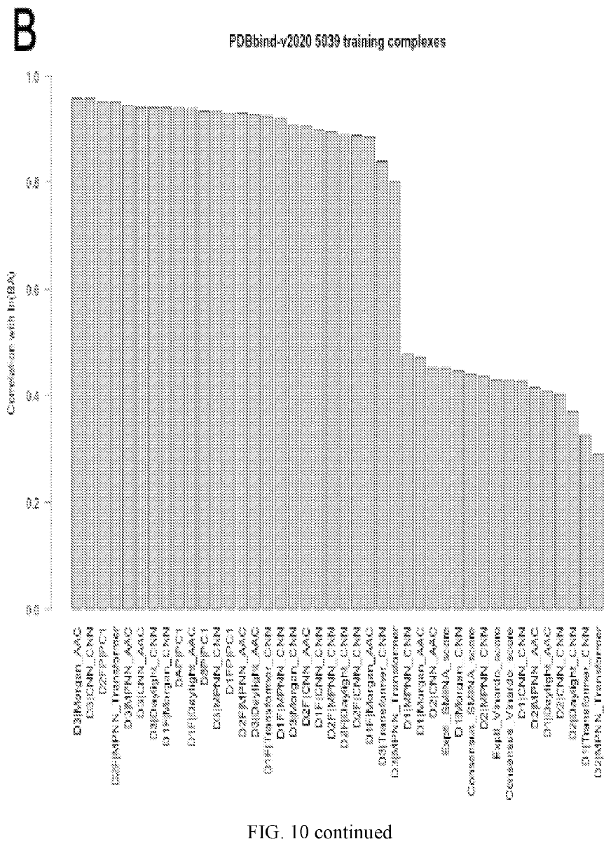

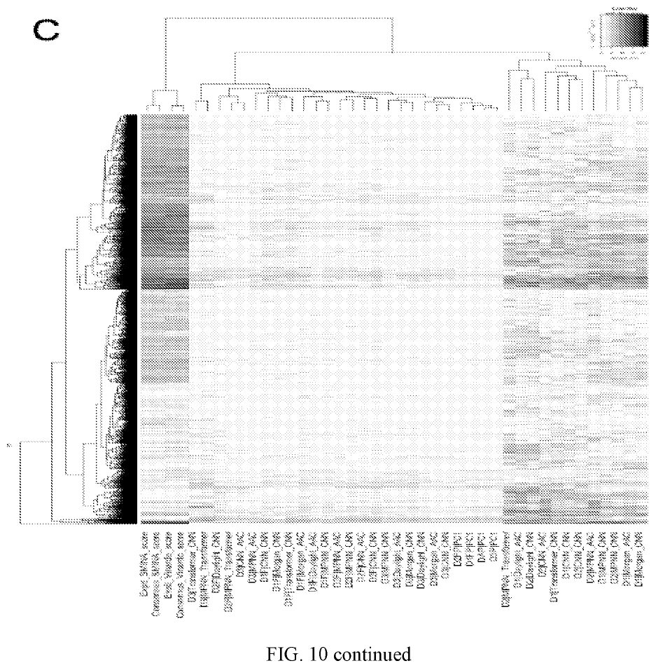

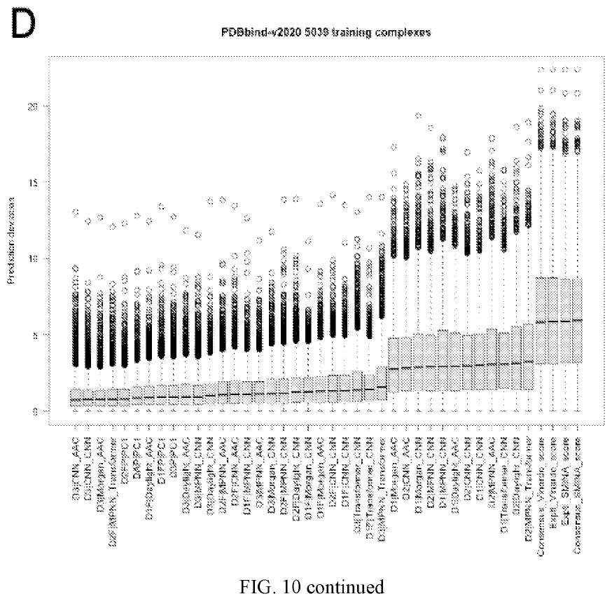

Given that the 38 DL models (6 D1, 6 D2, 10 D3, 6 DIF, 6 D2F, 1 D1FP, 1 D2FP, 1 D3P, and 1 DMP) as well as the 4 docking models were investigated, first their predictions by Pearson correlation values among themselves were compared. FIG. 4A shows a heatmap of Pearson correlation values for all pairs of predictions for the CoreSet. As expected, high correlations (i.e., similar predictions) are observed among (1) the 4 docking models and (2) the fine-tuned or PC1 models (DIF, D2F, D1FP, D2FP, D3P). The BindingDB-based models (D1 and D2) are less correlated among themselves. Correlations between the experimental binding affinity and those predictions show that the top model is D1F|Daylight_AAC (pre-trained and fine-tuned) and the next top 3 models are PC1 models (FIG. 4B). Another comparison analysis was also performed by prediction errors or deviations from the experimental binding affinity. FIGS. 4C and 4D show a heatmap and a box-whisker plot of prediction deviations or absolute errors for the 42 models. The fine-tuned or PC1 models tend to show lower errors than the BindingDB models, which in turn tend to show lower errors than the docking models. The top 5 models in terms of median errors are the 4 PC1 models and D2F|MPNN_Transformer (de novo-trained and fine-tuned). Similar but more pronounced correlations and deviations are observed for the training set, as expected (FIG. 10).

### Example 3: Meta-Models of Docking Tools and DL Models

Several versions of meta-models were run to cover the spectrum of input features discussed in methods (Table 1), with the goal of parsing the contributions of particular components of the models. The broad feature groups included docking scores, deep learning scores (untransformed), and deep learning scores (PC-transformed). Early exploratory analyses with SwissADME-derived ligand properties suggested the inclusion of molecular weight to contend with possible biases in the docking scores. Furthermore, the impact of different deep learning training sets and models was examined. The full training set, the RefinedSetCoreSet, consists of 5,032 complexes, while the benchmark test set, the CoreSet, consists of 266 complexes.

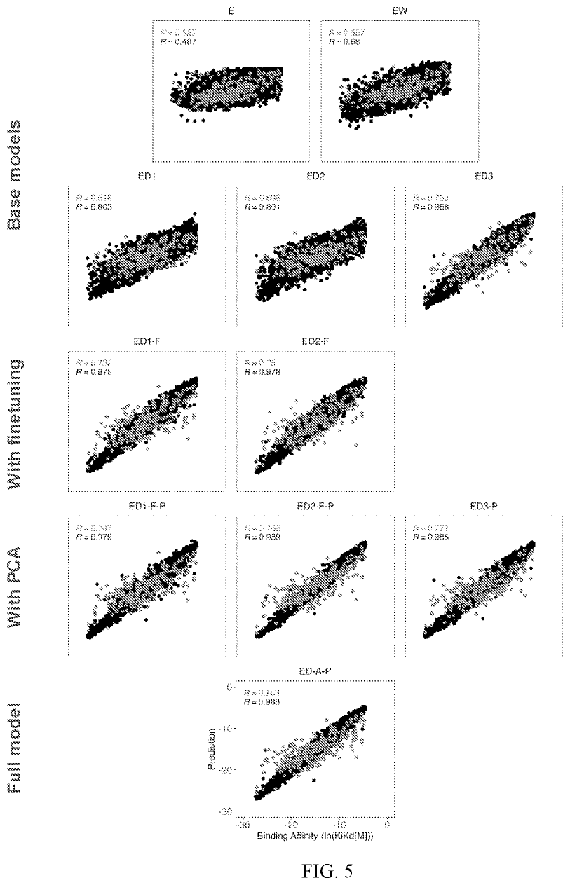

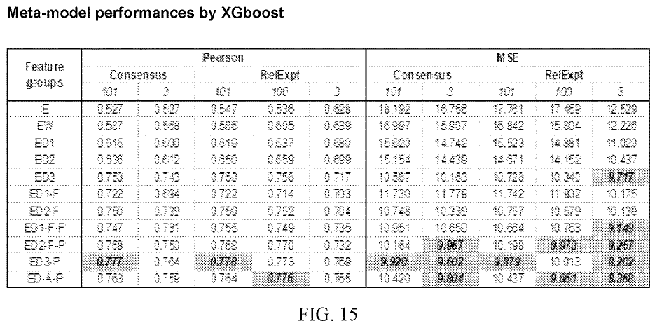

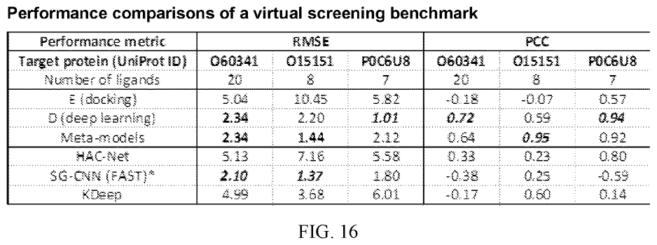

The representative meta-model results using XGBoost, which are presented in FIG. 15 and shown in FIG. 5, showed the best performance among the 4 algorithms. The different panels show the scores for the XGBoost meta-model trained on RefinedSetCoreSet in black and the corresponding test scores for the CoreSet in red. The panels of FIG. 5 are arranged in a manner that reflects the different sets of features and training data: starting with meta-models constructed entirely based on the docking methods, SMINA and Vinardo; adding in MW; the inclusion of DL scores based on BindingDB or PDBbind trainings; and the fine-tuning and PCA. In particular, the panels are arranged as follows:
The first row represents the meta-models constructed entirely based on the docking methods, SMINA and Vinardo. The first panel shows results for the two scores alone (E-Only), while the second panel shows the results also including the molecular weight parameter (E-MW).
The remaining rows show results for meta-models that include deep learning scores. The second, third and fourth rows represent successive processing of the deep learning model scores, with the second row showing the average scores (ED1, ED2, ED3), the third row (if a panel is present) showing the average after fine-tuning (F) using the RefinedSet\CoreSet (ED1-F, ED2-F), and the fourth row showing the scores using the optimal number of PCs from a PCA (P) on the previous level (either fine-tuned or not) (ED1-F-P, ED2-F-P, ED3-P);
The columns of the second, third and fourth rows show the three different base training sets used for the deep learning scores, where the leftmost column is based on the pre-trained models on BindingDB (ED1 series), the central column is based on the de-novo-trained models on BindingDB (ED2 series) and the rightmost column is based on the de-novo-trained models on PDBbind (ED3 series).
The single panel in the fifth and last row shows the results for the meta-model ED-All-PC, which is based on a PCA on all the training scores from ED1-F, ED2-F, and ED3.
   Note that, in the PC-based models, the number of PCs was obtained by maximizing the respective machine-learning scores for a leave-out validation set (the RefinedSet\CoreSet was split 80%-20% into training-validation partitions).

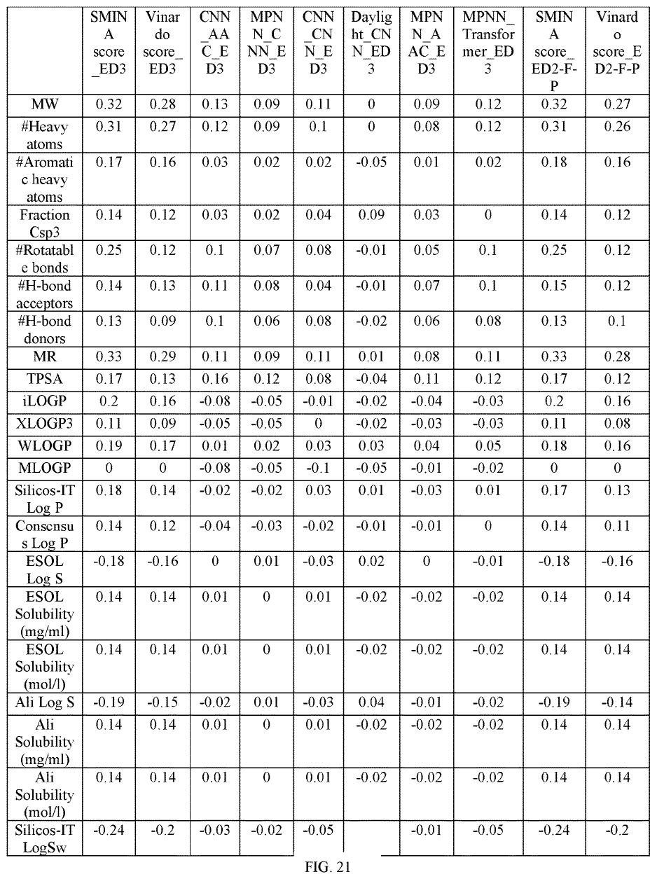

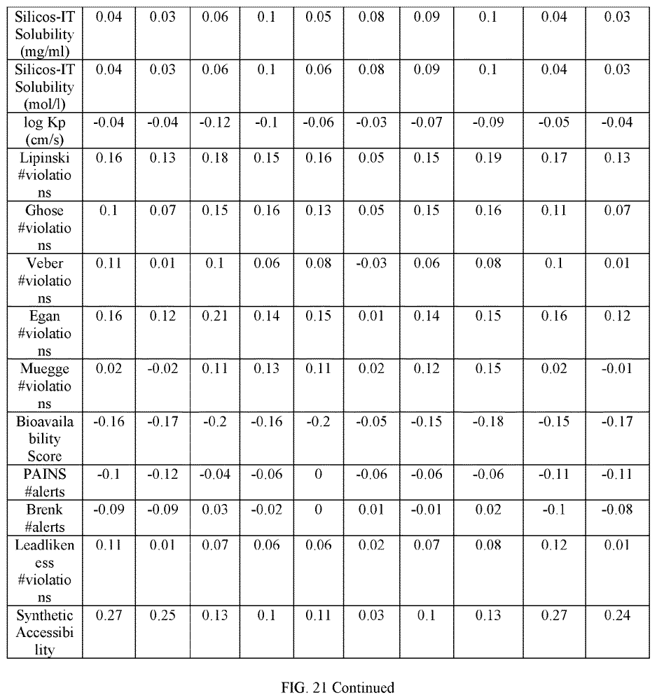

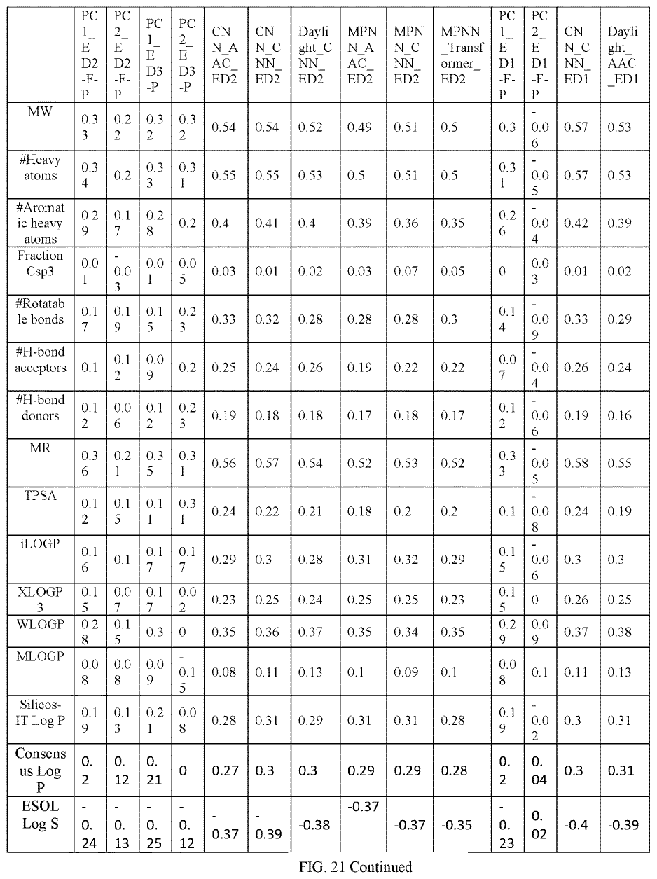

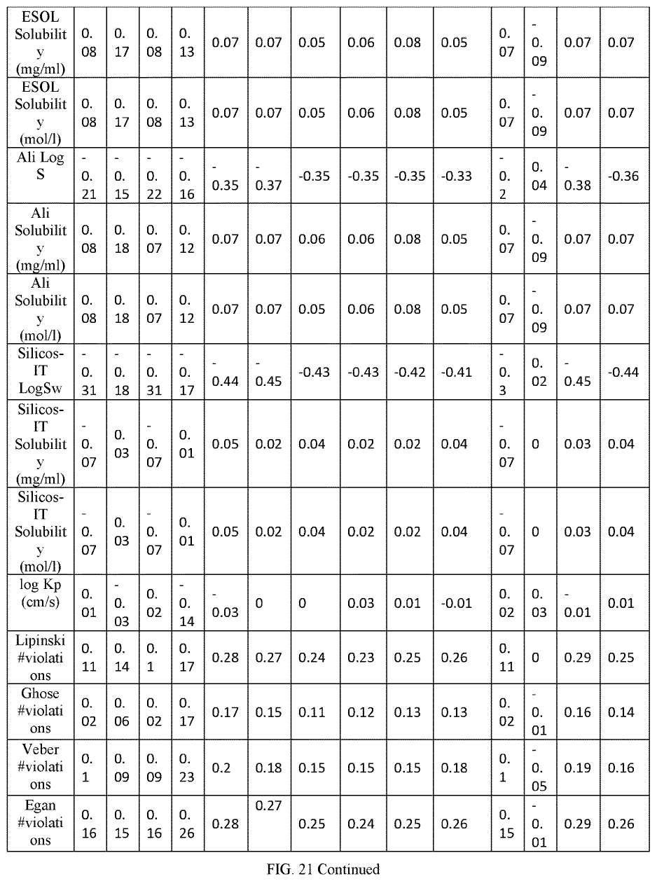

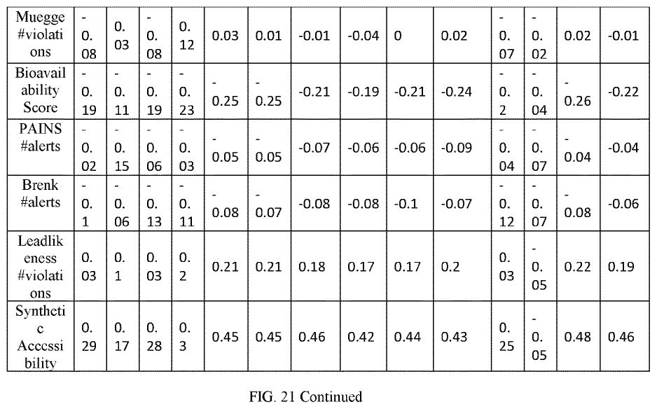

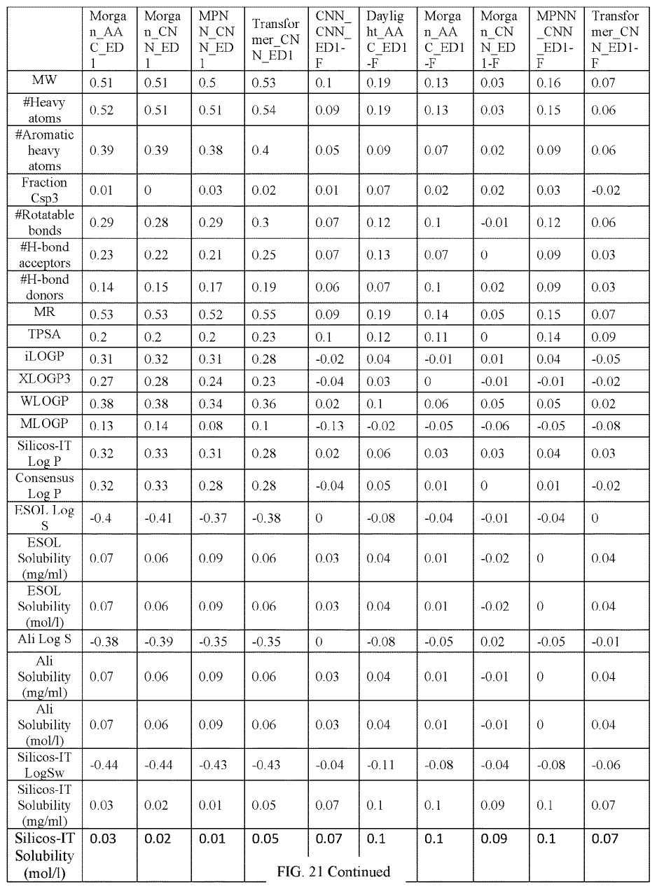

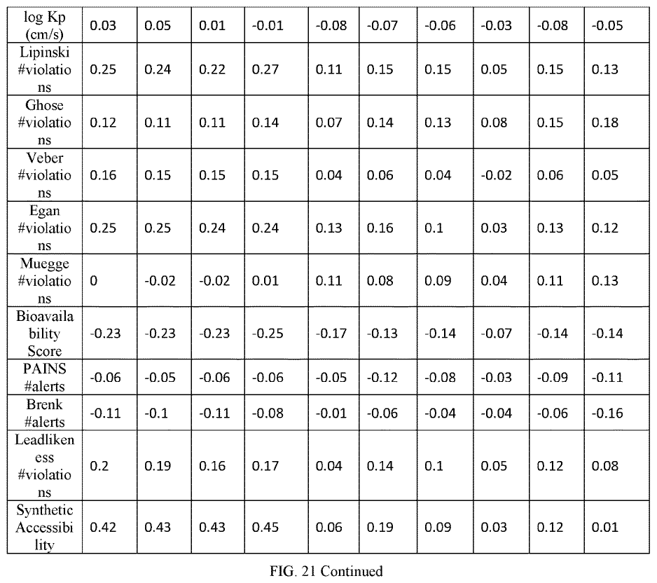

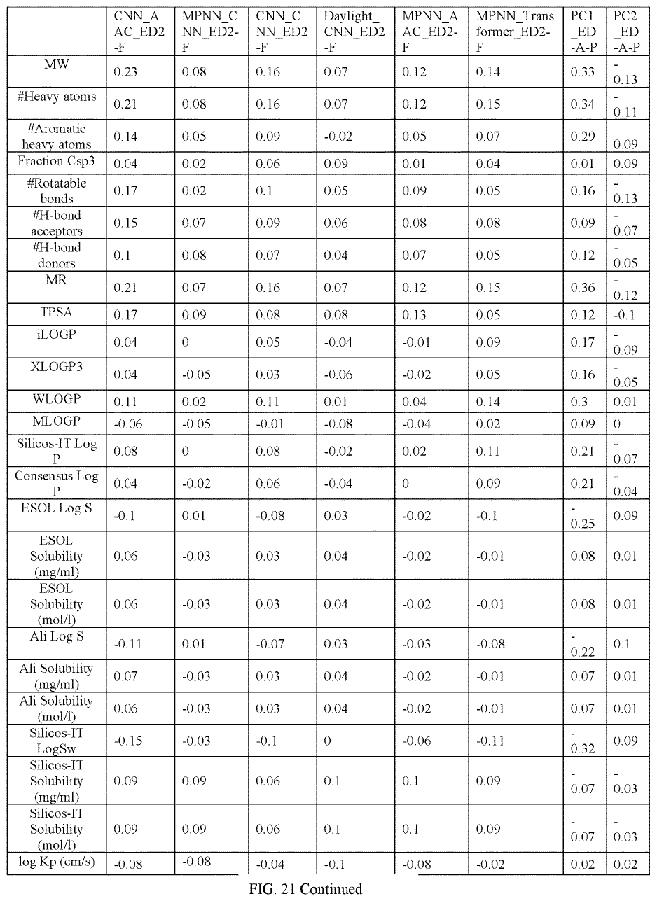

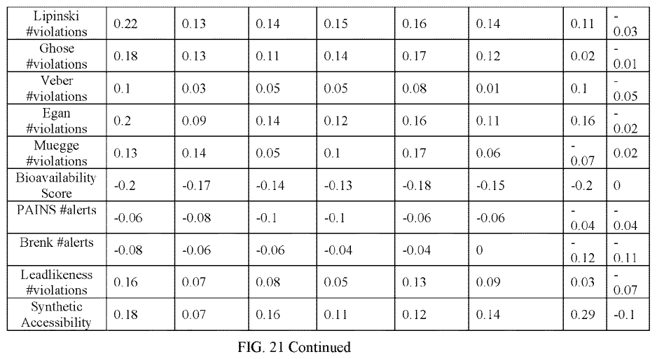

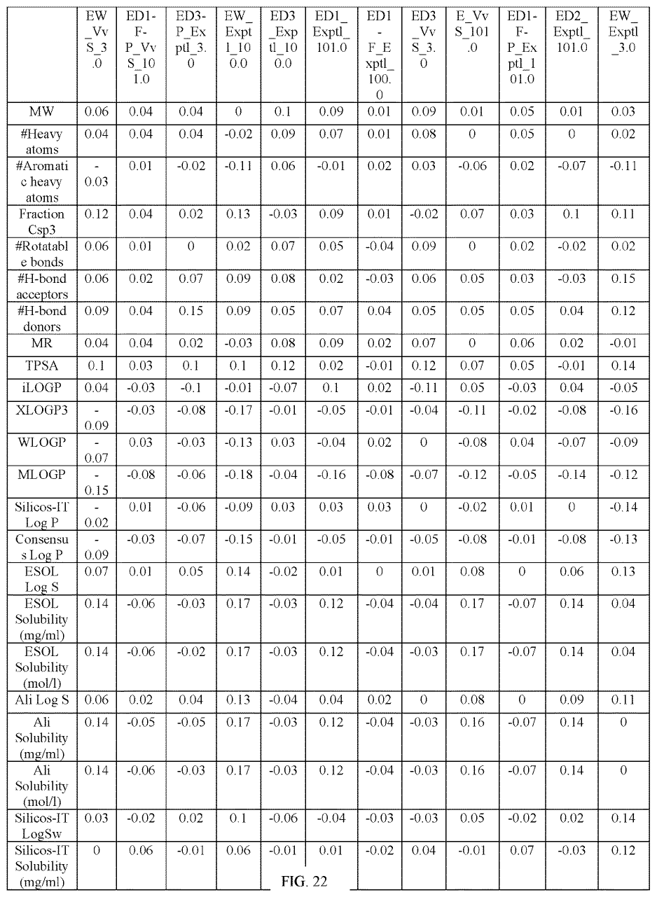

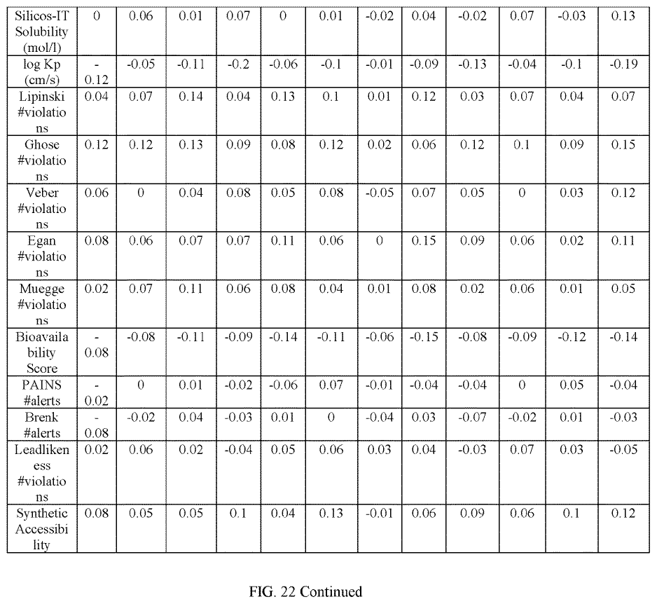

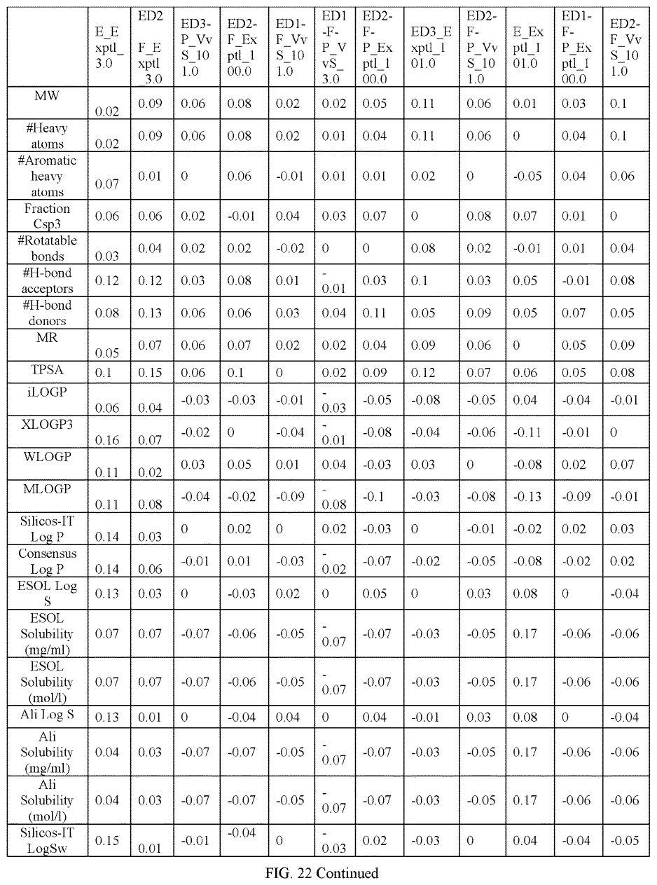

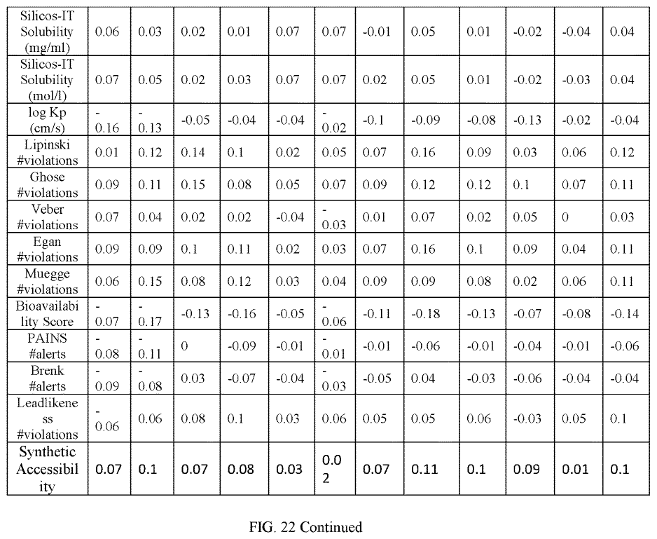

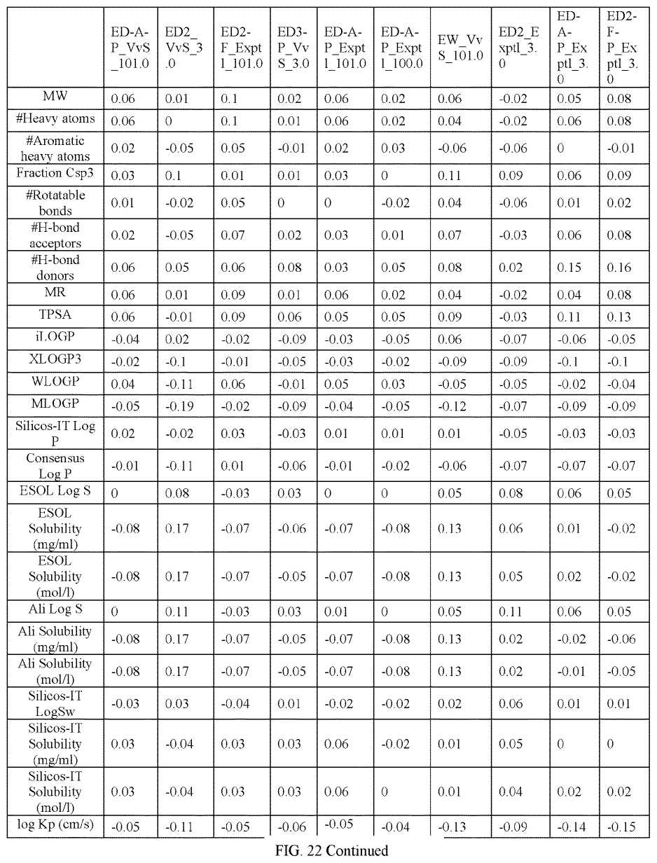

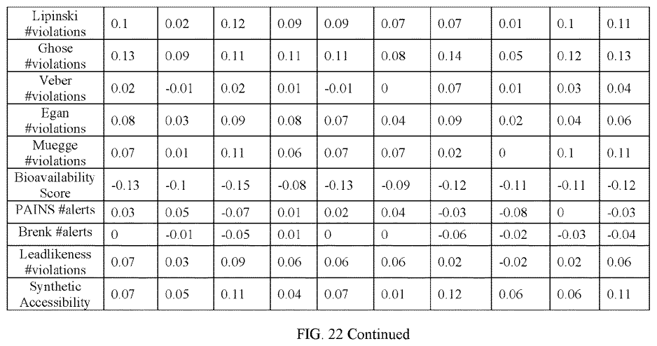

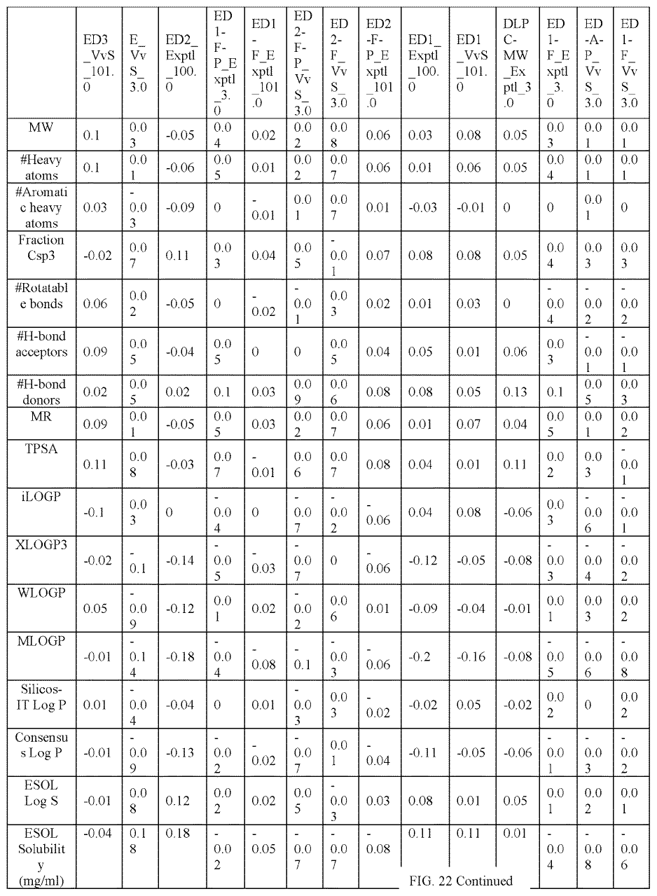

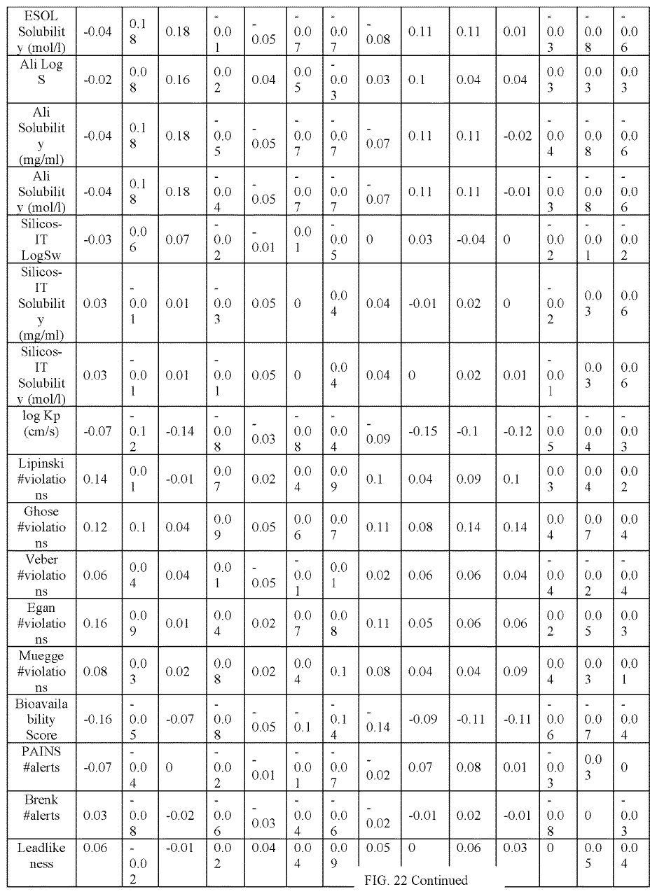

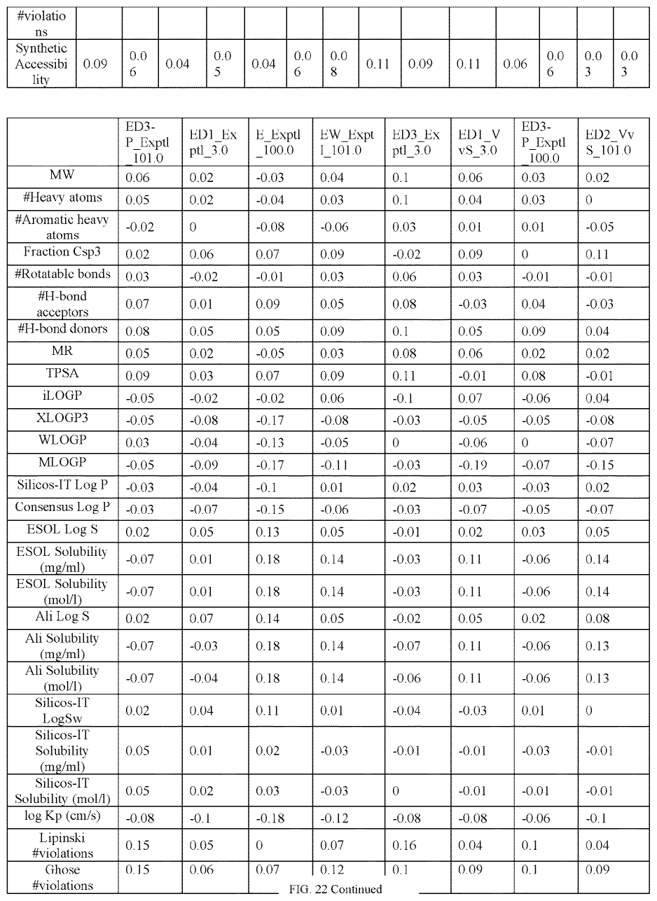

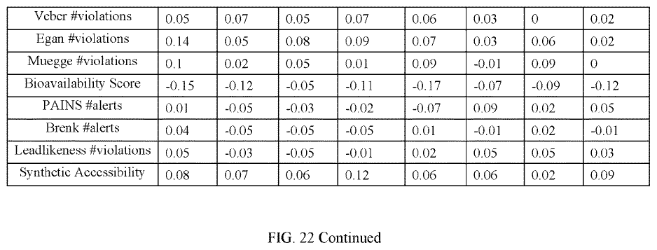

It is first noted that the docking results alone (E) produce a moderate Pearson correlation for the test set (0.527). Unexpectedly, the E correlation for the training set (0.487) is lower than that for the test set. This might be due to the more careful curation and filtering of complexes in the CoreSet, relative to the broader RefinedSet\CoreSet. Moreover, adding in the molecular weight of the ligands (EW) showed significant improvements for both training and test sets. The original motivation for including the MW was the fact that the absolute deviation between the SMINA and Vinardo predictions and the experimental binding affinity is relatively highly correlated (r~0.3) with the MW and a few related ADME features (such as the number of heavy atoms and molecular refractivity) than others (FIG. 21). Including MW was an attempt at addressing this bias in the docking scores. It was also noticed that the MW was correlated with the absolute deviation in the scores for some of the deep learning features, including the top principal component. When molecular weight was added as a meta-model feature (in all the meta-models except for E), it was found that the deviations of the final predictions are much less correlated with molecular weight (FIG. 22).

In the next rows, it can be seen that the meta-models including DL scores significantly increase the Pearson's correlation relative to the docking-based meta-models. The fine-tuning of the DL scores on the RefinedSet improves the scores relative to the BindingDB-trained models. PCA on any of the DL scores improves performance even more. However, it is noted that D3 was trained on the RefinedSet\CoreSet, while the D1-F and D2-F models were fine-tuned on the same set. This means that the training set for the deep learning training/fine-tuning process is identical to those for the associated meta-models (ED1-F, ED1-F-P, ED2-F, ED2-F-P, ED3, ED3-P, and ED-All-P). This also means that the “leave-out validation” split for the PC number hyperparameter optimization is not a completely independent dataset. The consequence of the most overlap of the two training sets is that an extremely high training set correlation for those meta-models was observed. Nonetheless, the CoreSet was not included in any of the deep learning training steps, and so the test set remains completely unseen until the prediction phase. The high performances of these models on the CoreSet therefore signal good overall model robustness.

Beneficial effects observed in the meta-models by performing PCA on the DL outputs are possibly due to the denoising or debiasing of outputs. The denoising is achieved by first running multiple parallel cross-validations, and then combining them into a lower-dimensional representation. However, unlike the DL results in FIGS. 3A and 3B, the performance improvement in meta-models is not observed with the first PC alone, but rather requires the combination of a few PCs. It is also noted that, among the 4 PCA-based models (ED1-FY-P, ED2-F-P, ED3-P, and ED-All-P), there are no large differences in the performance, i.e., different meta-model algorithms for PCA on different DL training processes show similar performances (R=0.76-0.78). This suggests that the information content of the constituent docking and DL model predictions have been maximized.

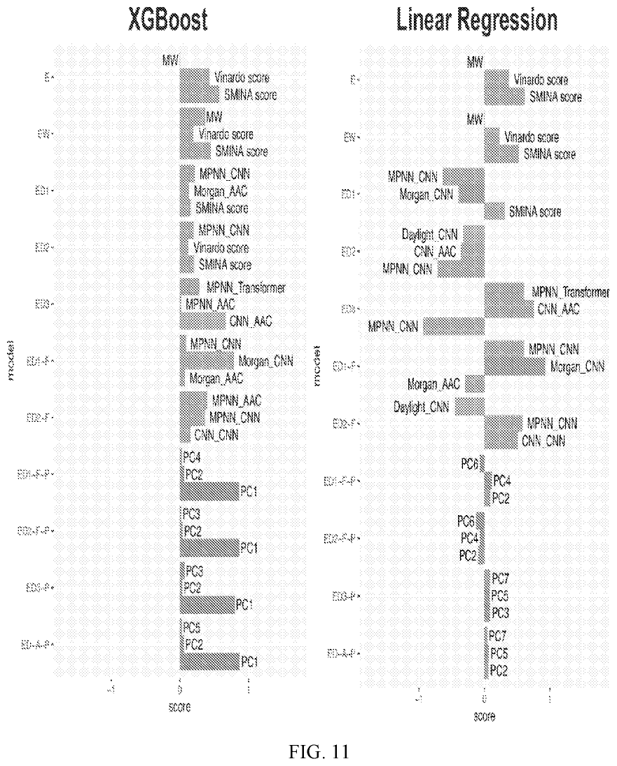

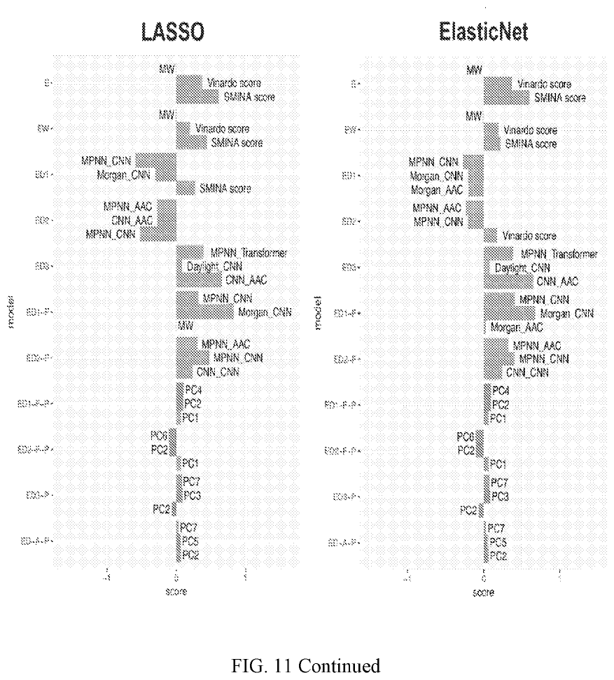

To understand the contributions of the component features, the feature importance scores for each meta-model were extracted (FIG. 11). It was found that, while the contributions of the docking scores dominate for the EW, ED1, and ED2, these contributions drop to very low values for the other DL-containing meta-models. In all meta-models, SMINA scores contribute more than Vinardo scores. However, given the aforementioned similarity between the SMINA and Vinardo scores, this difference in the contributions between the two docking scores is more likely to be an artifact of the modeling process (and how it deals with nearly collinear features). The other noteworthy aspect of the importance scores is that the performance of ED3, fine-tuned, and PC-based meta-models is dominated by one, or at most two significant features. ED1-F, ED2-F and ED3 are impacted mainly by two DL scores, while the PC models are dominated by PC1. It is again observed that PC1 alone is not sufficient to improve the predictions, but requires the assistance of other PCs. The patterns of feature importance scores, however, are not entirely consistent across the different ML meta-models. The top features of the LASSO and Linear Regression meta-models are distinct from the XGBoost top features. The signed feature importance scores in the linear models show less domination of single scores, especially in the PC-based models. In particular, PC1, which is individually highly correlated with experimental binding affinities, is less important in the predictions by the linear meta-models. Given the similar performances of the ML meta-models, it appears that there are several paths to attaining nearly equivalent overall correlation values.

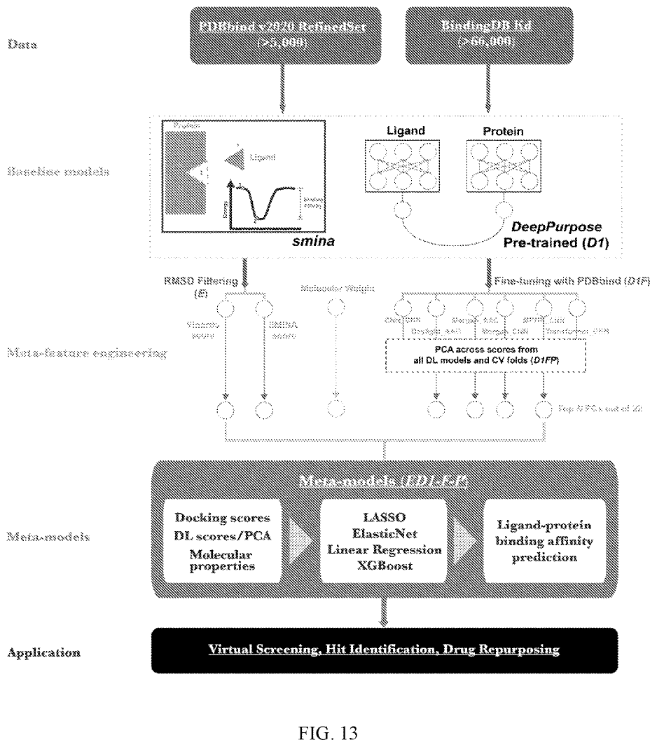

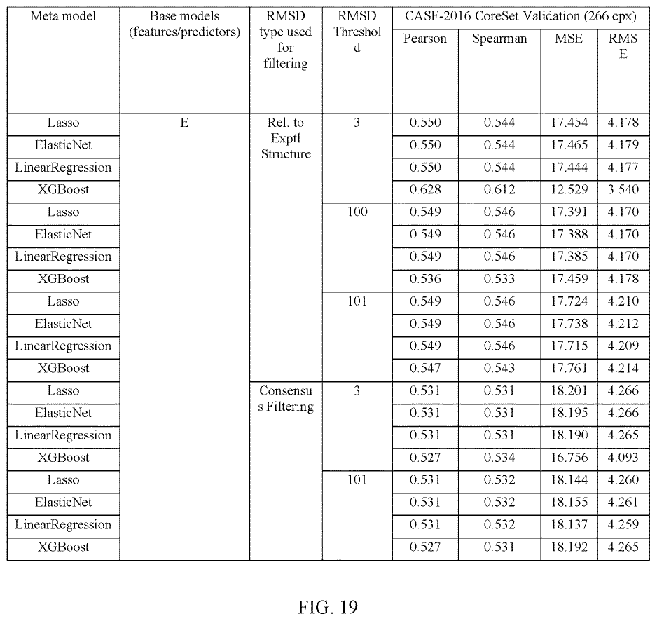

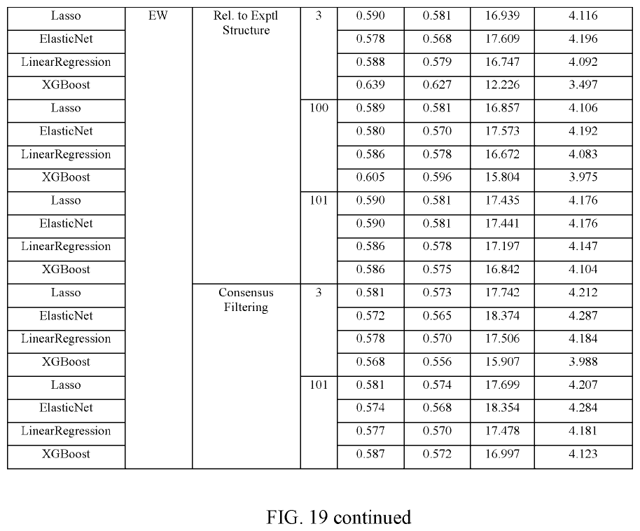

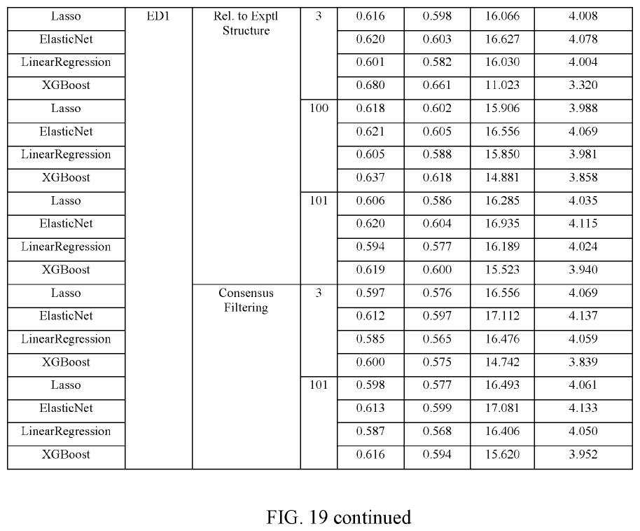

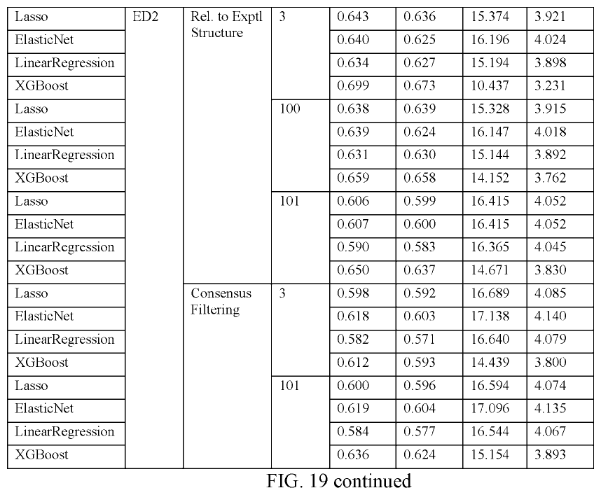

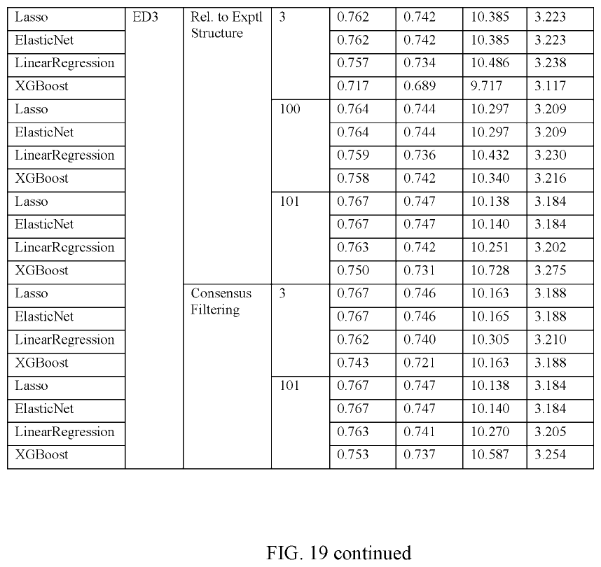

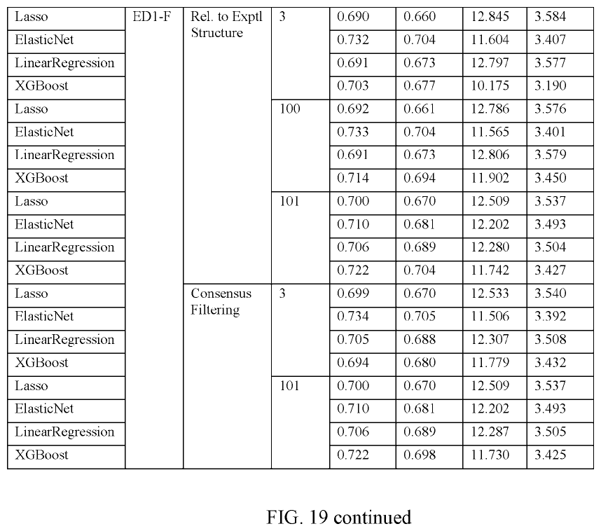

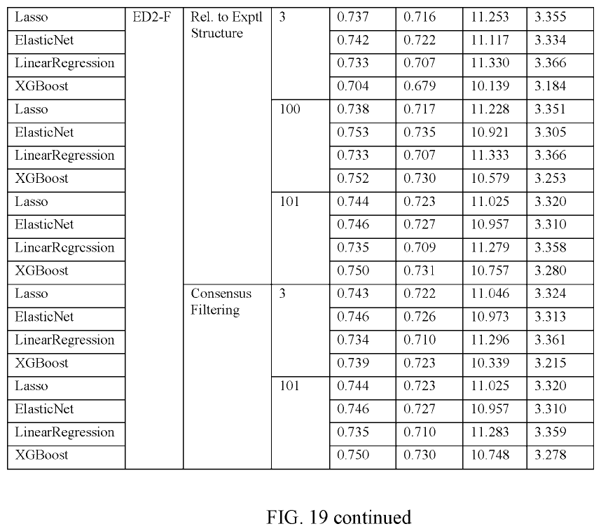

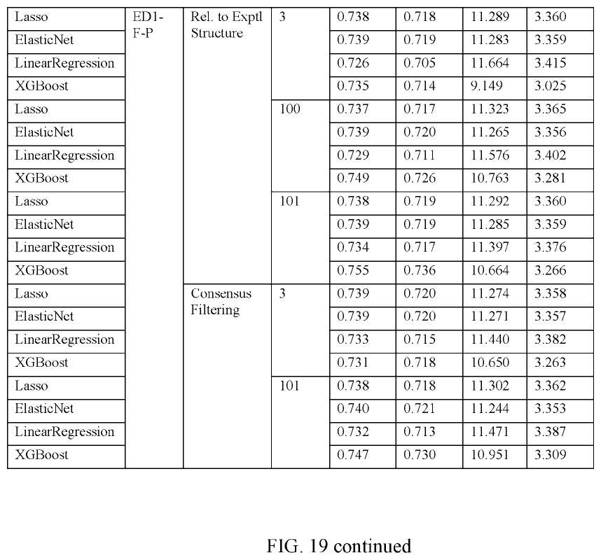

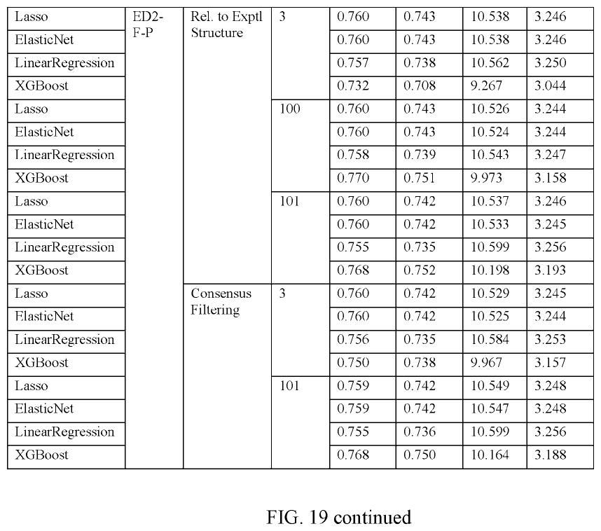

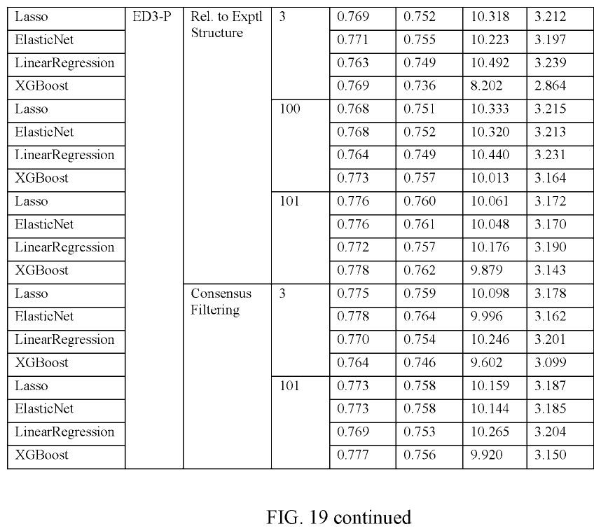

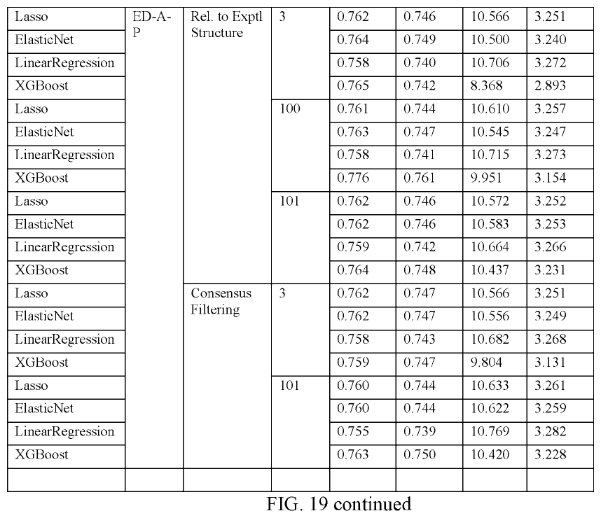

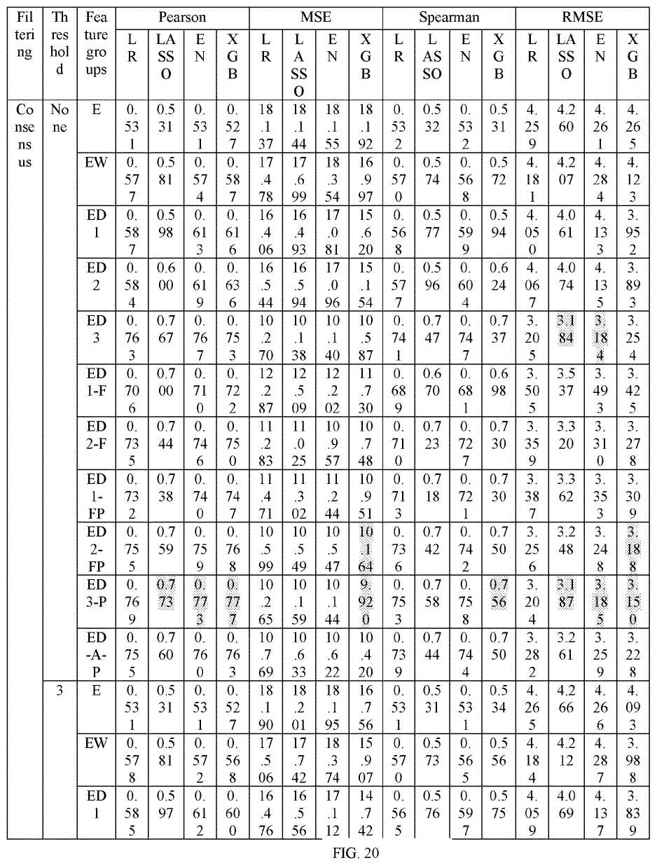

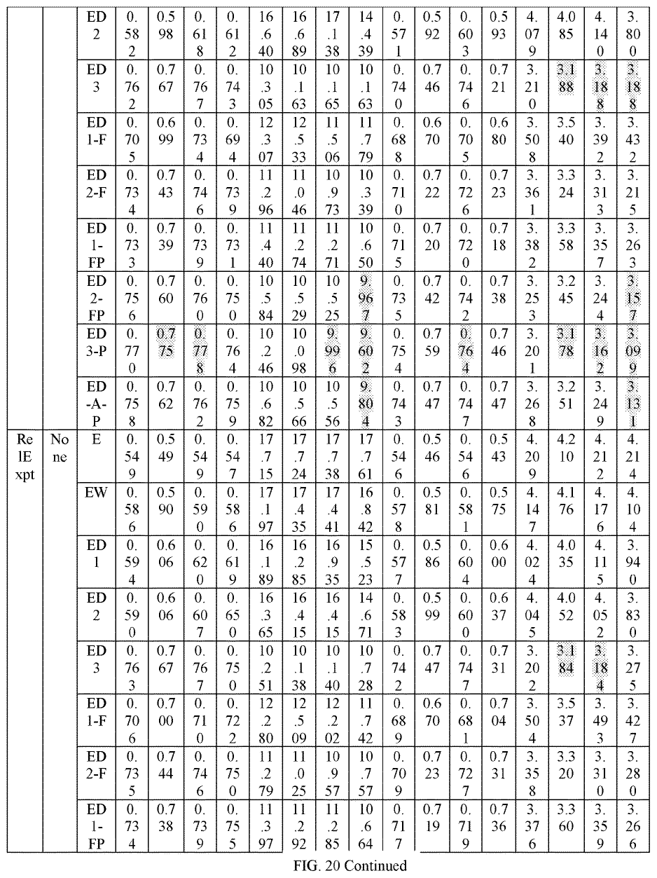

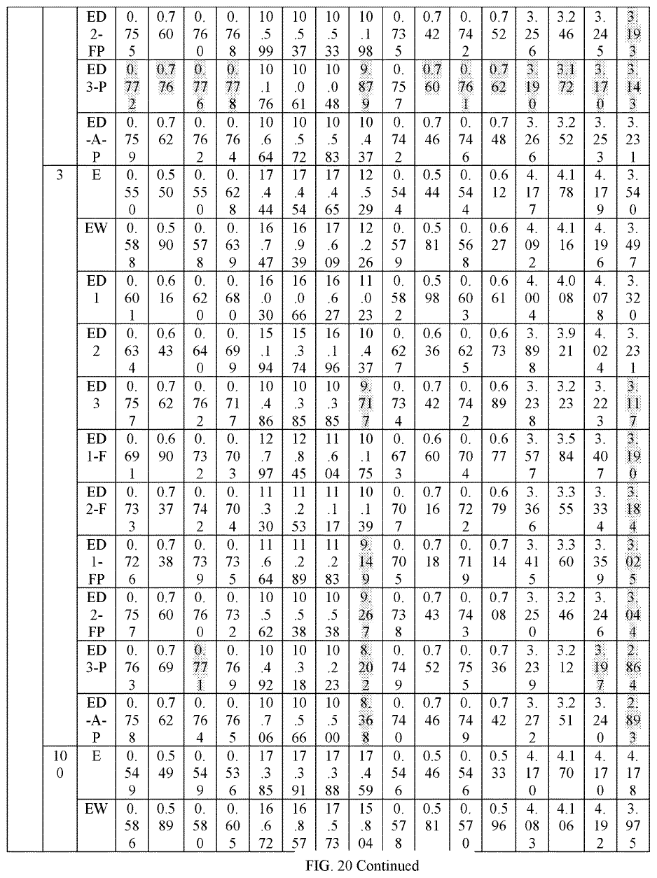

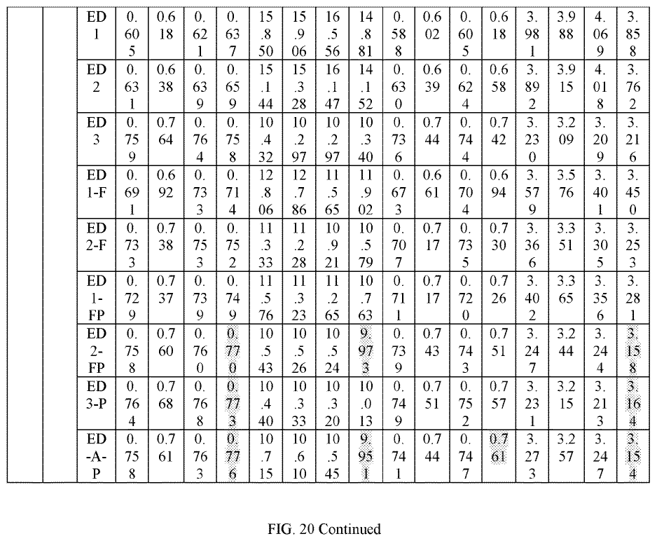

Given the improvement in the docking predictions by filtering out certain protein-ligand pairs (FIGS. 2 and 13), the effect of the Experimental and Consensus RMSD filters were explored with 3 and 100 Angstrom cutoffs on the meta-models (FIGS. 19 and 20.). It was found that the E and EW meta-models are affected by the different filters, with the Experimental RMSD filters producing slightly better results than the Consensus filters. This reinforces the idea that the docking affinity predictions benefit from constraining the predictions based on the pose. However, as the influence of the docking predictions decreases in the meta-models, it appears as if the impact of the RMSD filters also goes down and the best-performing meta-models show negligible difference in their performance with the various RMSD filters.

### Example 4: Prediction Synergy and Complementarity of Docking and DL Tools

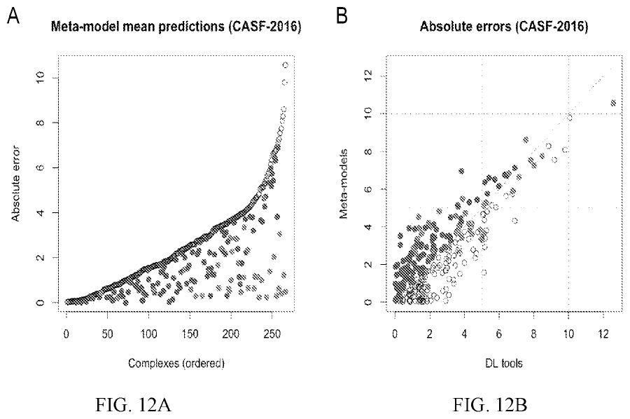

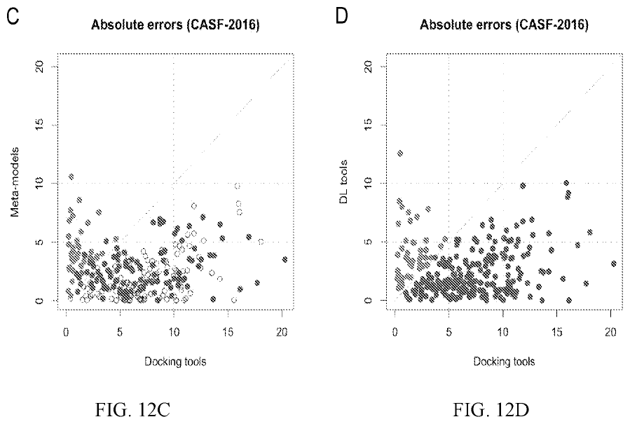

As better overall performance was obtained by meta-modeling of docking and DL tools, the degree of prediction synergy and complementarity of the two groups of tools, i.e., whether the meta-models utilize the strengths of both tool groups and the degree to which the two provide non-overlapping information, were further investigated. To do this, focus was on absolute errors of the mean predictions for the benchmark set by the 4 meta-models with ED-A-P, by the 4 docking tools, and by the DAP|PC1 model (FIG. 4D). When mutually compared, the mean predictions by the meta-models achieved the lowest errors for 123 of the 266 complexes (46.2%), the DAP|PC1 for 104 complexes (39.1%), and the docking tools for 39 complexes (14.7%) (FIGS. 12A-12C). If only the docking and DAP|PC1 tools are compared, the former showed better mean predictions for 48 of the 266 complexes (18.0%) than the latter (FIG. 12D). This suggests that, beyond the globally better performance of the meta-models, prediction synergy is realized for 46.2% of the benchmark set by the meta-modeling due to complementary information of the docking and DL tools (even though each group of tools is better at predicting a subset of complexes by itself). Similarly, although the DL tools are generally better than the docking tools, there exists a small subset of complexes that are better predicted by the latter.

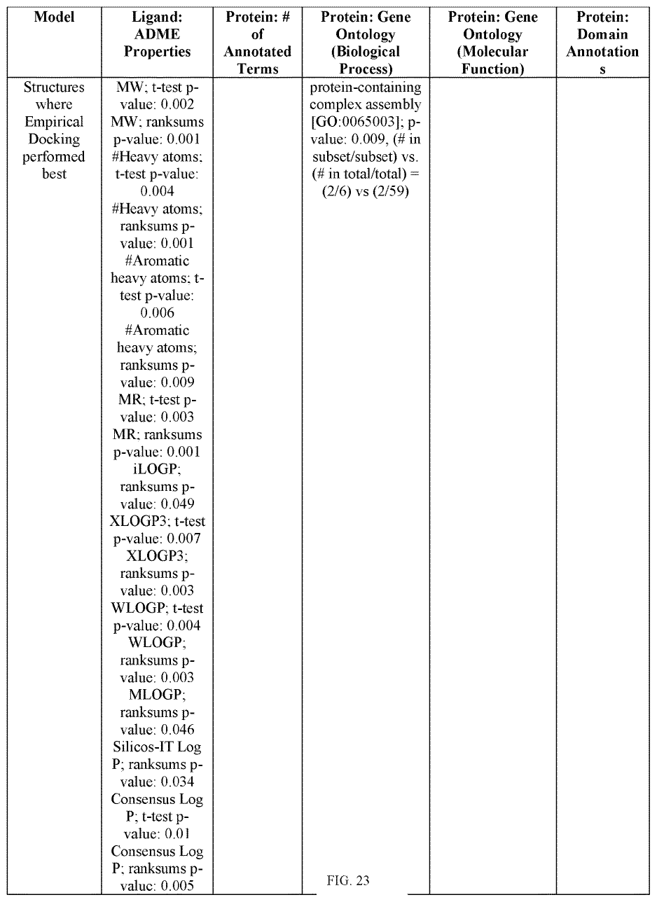

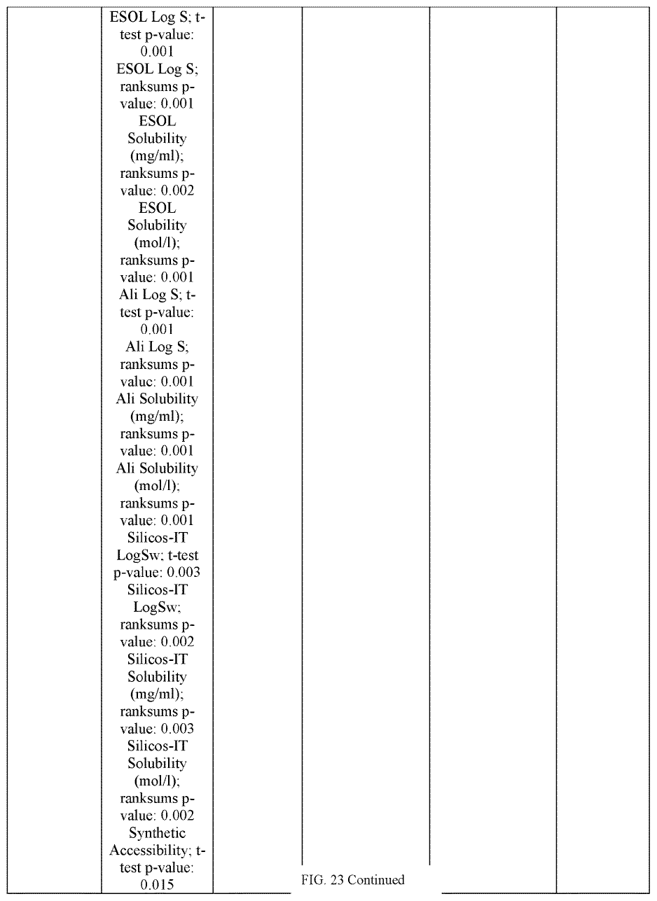

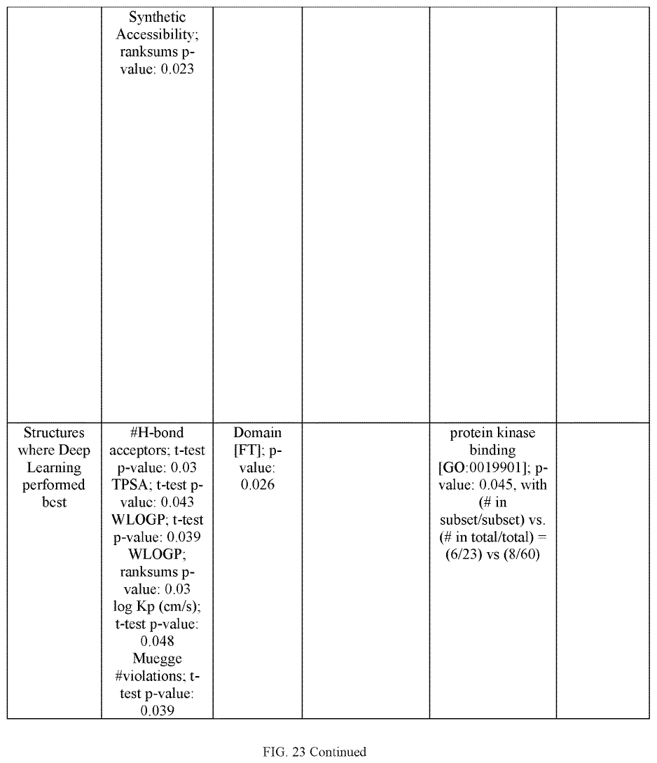

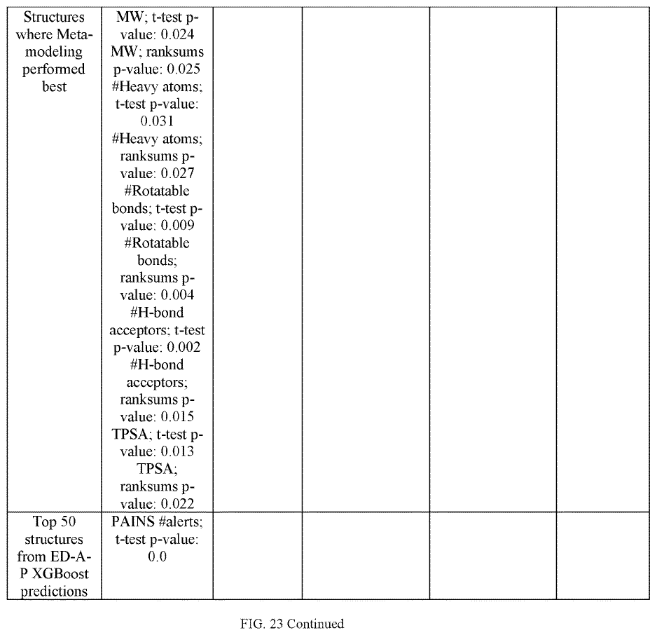

To further parse the differences between the outputs of the prediction tools, it was assessed whether certain properties of the proteins or ligands are over- or under-represented in the complexes which were: (1) Best modeled by empirical docking; (2) Best modeled by deep learning; (3) Best modeled by meta-modeling. Also explored were the top 50 complexes as predicted by the XGBoost Meta-model for ED-A-P to see if the performance of the meta-models relative to the experimental data was contingent on protein or ligand properties. Protein features were extracted from the UniProt database and ligand features from the SwissADME database. The significance of the occurrence of certain annotations (using Fisher's exact test) and of the distributions of certain quantitative features (using a two-sided Wilcoxon test) were tested for each of the four subsets of complexes. Specifically, it was considered whether several UniProt features were annotated more or less frequently, and whether certain Gene Ontology (GO), domain and pathway annotations occurred with different probabilities in the subsets. Also compared were the distributions of the ligand SwissADME features. The results (FIG. 23) indicate that there are practically no significant (nominal p-value 0.05) protein features prioritized in the four cases considered. This may be due to the fact that several proteins (and their associated UniProt IDs) are duplicated in the CoreSet. The number of reviewed proteins with UniProtIDs drops from 243 to 60 after removal of all duplicates. The highly significant results with nominal p-values <0.01 (both Wilcoxon and t-tests) are the distributions of several ligand features including MW, lipophilicity, and solubility measures in the structures for which the empirical docking tools perform the best. There is also some hint of significance for MW, the “#Rotatable bonds” and “#H-bond acceptors” in the structures for which the meta-models perform the best.

### Example 5: Comparison with Structure-Based DL Tools

Having built the meta-models based on sequence-based DL models, comparisons with structure-based DL tools through voxelization of binding pockets was of interest. The following 3 tools were focused on by re-training them with the training data used herein for fair comparisons as much as possible.

### HAC-Net (Kyro et al. 2023)

A recent study developed a new structure-based DL ensemble or meta model, HAC-Net, by combining MP-GNN (message passing graph neural networks) and 3D-CNN (convolutional neural networks) trained on the PDBbind-2020 general set of >18,800 ligand-protein complexes. Their best performance was a Pearson correlation of 0.846 for the external data of the PDBbind-2016 CoreSet of 290 complexes. The focus is on the PDBbind-2020 RefinedSet rather than the general set because of two reasons: (1) higher quality and (2) limited resources. For a fairer, but limited, comparison, HAC-Net was re-trained with the training set of the PDBbind-2020 RefinedSet-CoreSet by subsetting from their train/validation/test data splits (4942/82/265 out of 18818/300/290 complexes from the general set, respectively). The Pearson correlation for the test set is 0.756, which is worse than the best meta-model performance of 0.777. Their MSE is not comparable to the one presented here because they used −log 10(Kd/Ki) while in this study ln(Kd/Ki) was used.

### FAST (Jones et al. 2021)

Jones et al. developed meta or fusion models, FAST, by combining 3D-CNN and SG-CNN (spatial graph CNN). Given technical difficulties, it was possible to run their pre-trained SG-CNN model trained on the PDBbind-2016 general set excluding the CoreSet. The model was tested against the CoreSet of 266 complexes used herein, which resulted in a Pearson correlation of 0.716, worse than HAC-Net. Their MSE is not comparable to the present invention for the same reason above.

### KDeep (Jimenez et al. 2018)

KDeep was one of the earlier structure-based DL models with 3D-CNN to predict ligand-protein binding affinity. Their reported Pearson correlation on the PDBbind-2016 CoreSet was 0.82, which was not verifyable because their code is not open-source. The KDeep model available on the public web-server could be re-trained with limited data size (KdeepTrainer, playmolecule.com/KdeepTrainer/). While it was not possible to use full training data of the present Example due to the limitation, it was possible to re-train the KDeep model for 250 epochs with each of 3 training sets of random 1,800 complexes sampled from training set of the PDBbind-2020 RefinedSet. Next the 3 trained models were tested against the CoreSet used herein. Pearson correlations of 0.718-0.766 were obtained, which are similar to those reported values of 0.701-0.738 by Kwon et al. using the PDBbind-2016 RefinedSet as a training set and 4 different learning rates.

Despite those limited comparisons, it could be concluded that the meta-models or the sequence-based DL models described herein show competitive performances to more sophisticated structure-based DL models. (Kwon, Y. et al; International Journal of Molecular Sciences 2020, 21 (22), 8424)

### Virtual Screening Benchmark

To further explore the performance of the models on specific target proteins with multiple ligands, a virtual screening benchmark was designed against 3 target proteins for 35 complexes from the PBDbind v2020 GeneralSet: O60341, O15151, and P0C6U8). The performances of models for the 3 target proteins are shown in FIG. 16 in comparison with the 3 tools described above, HAC-Net (re-trained), SG-CNN (FAST; pre-trained), and KDeep (the up-to-date default version on their web server). The pre-trained SG-CNN performed best for O60341 and O15151 in terms of RMSE (2.10 and 1.37, respectively). This was expected because their training data were the PDBbind v2016 GeneralSet, which includes 30 out of the 35 complexes in the benchmark set except 5 complexes for 060341. Otherwise, the meta-models or fine-tuned DL models performed best for all 3 target proteins in terms of both RMSE and Pearson's correlation. The best performing models are ED2-F_VvS_101.0_LinReg (meta-model; docking+MW+de novo-trained & fined-tuned DL by linear regression) and D1F|Daylight_AAC (pre-trained & fine-tuned DL model) for O60341 (RMSE=2.34), EW_VvS_101.0_LinReg (meta-model; docking+MW by linear regression) for O15151 (RMSE=1.44), and D2F|MPNN-AAC (de novo-trained & fine-tuned DL model) for P0C6U8 (RMSE=1.01). The Pearson correlation metric is considered as less reliable due to the small sample size.

As noted in the previous Example on prediction synergy, different meta-models or component DL/docking models may perform best for different complexes or proteins. In the case of this virtual screening, it was noticed that while some of the same meta-models would often predict the binding affinities for 060341 and P0C6U8 with reasonable accuracy, they would not predict the affinities for 015151 as well, and vice versa. For example, ED2-F_VvS_101.0_LinReg showed low RMSEs for 060341 (RMSE=2.34) and P0C6U8 (RMSE=2.54) but higher RMSEs for 015151 (RMSE=5.88). In particular, it is observed that 6 out of the 8 ligands in the complexes for 015151 were k-mers (k=10, 12, or 15) with relatively high MW of >1000. Among the 35 complexes there are 18 k-mers with mean MW of 927.2 compared to 762.7 for 17 non-k-mers. It is noted that about 5% of the RefinedSet (used for model training) and about 10% of the GeneralSet contain k-mers (up to 10-mer and 19-mer, respectively). Given that k-mers are not representative and tend to have high MW, the aim was to assess whether high or low MW causes differences in the performance of the meta-models independently from the choice of a target protein by dividing ligands into two groups of low MW<=900 and high MW>900 (the median threshold for the 35 complexes). By focusing on ED2-F_VvS_101.0_LinReg from above, it was found that the accuracy for low MW ligands is better than high MW ligands (RMSE=2.45 vs. 4.36). This is expected given the fact that the ligands in the RefinedSet used for training have mean MW of 384.3 and standard deviation of 172.6 and that the Pearson correlations between predictions and MW are negative ranging from −0.62 to −0.29. These results suggest that there might be identifiable features of ligands and/or proteins which would explain why certain meta-models perform variably for different target proteins.

Overall, it was concluded that the family of the models presented herein are competitive to several state-of-the-art structure-based models and also useful for a virtual screening application.

### Discussion of Examples

The framework presented here is based on the concept that different models designed and trained for the same prediction task often carry model-specific biases, and so combining the results of their independent predictions may yield superior results if the biases are even partially independent and the models are complementary to some degree. Indeed, this was borne out in the protein-ligand binding-affinity-prediction task under consideration in several ways. First, it was found that combining the predictions from empirical docking methods with those from the pre-trained or de novo-trained deep learning tools can yield gains in predictive power over the individual approaches (ED1-3 vs. E-Only or D1-2). Second, utilizing models trained on BindingDB as the base and refining them on the PDBbind RefinedSet resulted in significant improvements (D1 and D2 vs. D1-F and D2-F, respectively), demonstrating that fine-tuning pre-trained models could retain good predictive quality on both primary (BindingDB) and secondary (PDBbind) datasets (FIG. 8). Third, even within the suite of deep learning tools, transforming their prediction scores through principal component analysis could improve the performance on the test set. This may be due to a de-noising of individual DL scores. Finally, the use of meta-models enables one to explicitly add features that may be associated with biases in the individual models. For example, it was found that, for both empirical docking tools and some deep learning models, the deviation of the predictions from the experimental affinities was correlated with molecular weight. Explicitly adding in MW resulted in the deviations in the final predictions being significantly less correlated with molecular weight.

The machine-learning-based meta-models allow for the flexibility of adding or removing any component tools or physicochemical properties as a part of feature engineering. Models trained on multiple datasets (e.g. BindingDB and PDBbind) and on multiple data types (e.g. sequence and/or structure) may be unified to reduce individual biases. Furthermore, the DL models from DeepPurpose considered herein, being sequence-based, offer better scalability. Given that only protein and ligand sequences are required for the prediction, and not all-atom 3D structures, it is expected that increasingly large databases could be used for training in future iterations by running high-throughput experimental binding affinity assays (without the need for concurrent X-ray or NMR experiments) as a filtering strategy in early drug discovery. Considering that the observed performances of these models for both benchmarks of CASF-2016 and the small-scale virtual screening are better or on par with more sophisticated structure-based DL models, there is an argument to be made in favor of more scalable approaches. Additionally, it is noted that the public and private experimental binding affinity databases being currently used are highly fragmented, in that they are produced separately, with potentially different types of assays or at different scales. While efforts such as the PDBbind database manage to carefully curate a high-quality single dataset, not all data sources can be incorporated because of the type of data (e.g. experimental data without associated structures being available) or because of data access (e.g. behind a private paywall). The meta-modeling described herein can address some of these issues by pooling only prediction outputs from models trained on diverse public or private databases with or without available structures, protecting the databases themselves if necessary and maintaining performance quality. Subsequent in-depth analysis could also identify prediction synergy and complementarity of all available tools as demonstrated in FIG. 12. Finally, there might be value in expanding families of meta-models with different input data, each family being optimized to target a particular protein or class of proteins and/or ligands as demonstrated in FIG. 16.

This proof-of-concept study of meta-modeling can be readily extended to integrate with (i.e., to add more model features of) predictions from any other tools, such as HAC-Net, FAST, KDeep, and/or PerSpect ML, molecular properties or embeddings, or even predictions from the same base models trained on different orthogonal datasets, to address model bias and robustness, which is the key conceptual advantage of any ensemble or fusion model. In parallel to this type of horizontal extension, it is believed that meta-models may be expanded or stacked vertically, leading to deep meta-models. The meta-modeling approach offers promising and flexible strategies for future model iterations to further improve ligand-protein binding affinity prediction.

### Enumerated Embodiments

The following exemplary embodiments are provided, the numbering of which is not to be construed as designating levels of importance:

Embodiment 1 provides a method of training an algorithm to predict ligand-protein binding affinity, the method comprising:
generating at least one docking score for the binding affinity of one or more ligand-protein complexes using one or more structure-based docking tools, wherein the generating of the at least one docking score comprises the steps of:
selecting a receptor structure file;
generating docking poses and associated binding affinity scores for each of the one or more ligand-protein complexes by one or more docking tools
analyzing the resulting docking poses for the one or more ligand-protein complexes; and
generating at least one deep learning (DL) score for the one or more ligand-protein complexes using one or more sequence-based DL models, wherein the generating of the at least one deep learning score comprises the steps of:
selecting at least one DL model to predict binding affinities of ligand-protein complexes; and
generating scores for the one or more ligand-protein complexes using the at least one DL model;
generating a meta-model training dataset from scores by at least two of docking tools or DL models and at least one physicochemical property of the ligand or the protein; and
training a machine learning meta-model on the training dataset to generate an algorithm for predicting ligand-protein binding affinities.

Embodiment 2 provides the method according to embodiment 1, wherein the generating at least one docking score for the binding affinity comprises employing at least two empirical scoring functions (ESFs) in tandem.

Embodiment 3 provides the method according to any one of embodiments 1-2, wherein the at least two ESFs are SMINA and Vinardo.

Embodiment 4 provides the method according to any one of embodiments 1-3, further comprising preprocessing the docking scores for the one or more ligand poses in predicting each ligand-protein complex.

Embodiment 5 provides the method according to any one of embodiments 1-4, wherein the preprocessing comprises filtering the ligand poses based upon the experimental and/or predicted docking poses.

Embodiment 6 provides the method according to any one of embodiments 1-5, wherein the filtering comprises applying a root-mean-squared deviation (RMSD) filter.

Embodiment 7 provides the method according to any one of embodiments 1-6, wherein the filtering comprises at least one of experimental pose filtering or consensus pose filtering.

Embodiment 8 provides the method according to any one of embodiments 1-7, wherein the experimental pose filtering comprises calculating a deviation for at least one of the docking poses predicted by the ESF relative to an experimental structure of the ligand.

Embodiment 9 provides the method according to any one of embodiments 1-8, wherein the consensus pose filtering comprises calculating a deviation between the docking poses predicted by one of the ESFs relative to the docking pose predicted by another one of the ESFs.

Embodiment 10 provides the method according to any one of embodiments 1-9, wherein the filtering cut-off is about 3 Angstrom.

Embodiment 11 provides the method according to any one of embodiments 1-10, wherein selecting the at least one DL model comprises:
inputting ligand-encoding and receptor-encoding embeddings into one or more DL models;
determining a performance of the one or more DL models; and
selecting the at least one DL model based upon the performance of the one or more DL models.

Embodiment 12 provides the method according to any one of embodiments 1-11, wherein the at least one DL model includes at least one of a pre-trained model or a de novo-trained model.

Embodiment 13 provides the method according to any one of embodiments 1-12, wherein the at least one DL model is either pre-trained or de novo-trained using BindingDB.

Embodiment 14 provides the method according to any one of embodiments 1-13, wherein the at least one DL model is de novo-trained using PDBbind data.

Embodiment 15 provides the method according to any one of embodiments 1-14, wherein the at least one DL model is selected from the group consisting of CNN-CNN, Daylight-AAC, Morgan-AAC, Morgan-CNN, MPNN-CNN, Transformer-CNN, MPNN-Quasi-seq, CNN/RNN-CNN/RNN, CNN-AAC, MPNN-Transformer, MPNN-PseudoAAC, MPNN-AAC, and MPNN-CNN/RNN.

Embodiment 16 provides the method according to any one of embodiments 1-15, wherein the at least one pre-trained DL model is selected from the group consisting of CNN-CNN, Daylight-AAC, Morgan-AAC, Morgan-CNN, MPNN-CNN, and Transformer-CNN.

Embodiment 17 provides the method according to any one of embodiments 1-16, wherein the at least one de novo-trained DL model is selected from the group consisting of CNN-AAC, MPNN-CNN, CNN-CNN, Daylight-CNN, MPNN-AAC, and MPNN-Transformer.

Embodiment 18 provides the method according to any one of embodiments 1-17, wherein the at least one de novo-trained DL model is selected from the group consisting of CNN-AAC, MPNN-CNN, CNN-CNN, Daylight-CNN, MPNN-AAC, MPNN-Transformer, Daylight-AAC, Morgan-AAC, Morgan-CNN, and Transformer-CNN.

Embodiment 19 provides the method according to any one of embodiments 1-18, wherein the at least one pre-trained or the de-novo-trained model is fine-tuned using PDBbind data.

Embodiment 20 provides the method according to any one of embodiments 1-19, wherein the at least one or more DL models are either de novo-trained or fine-tuned using 10 repetitions of 5-fold cross-validations (CVs).

Embodiment 21 provides the method according to any one of embodiments 1-20, wherein principal component analysis (PCA) is performed on the DL scores from all CVs.

Embodiment 22 provides the method according to any one of embodiments 1-21, wherein prediction scores are generated from the first principal components of PCA.

Embodiment 23 provides the method according to any one of embodiments 1-22, wherein the ligand-encoding and the receptor-encoding embeddings is from DeepPurpose library.

Embodiment 24 provides the method according to any one of embodiments 1-23, wherein the meta-model is a linear model or a non-linear model and is selected from the group consisting of LASSO model, ElasticNet model, Linear Regression model, and XGBoost model.

Embodiment 25 provides the method according to any one of embodiments 1-24, wherein the meta-model training dataset further includes molecular weight of the at least two or more ligands.

Embodiment 26 provides the method according to any one of embodiments 1-25, wherein the selecting comprises selecting the DL model that achieves the performance of mean-squared-error (MSE)<0.6.

Embodiment 27 provides an apparatus for predicting a ligand-protein binding affinity, the apparatus comprising:
a processor;
a memory unit, and
a communication interface;
wherein the processor is connected to the memory unit and the communication interface; and
wherein the processor and memory are configured to implement the algorithm trained according to any one of the previous claims.

Embodiment 28 provides a non-transitory computer readable storage medium, storing computer-executable instructions for performing the method of any one of embodiments 1-26.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of training an algorithm to predict ligand-protein binding affinity, the method comprising:
generating at least one docking score for the binding affinity of one or more ligand-protein complexes using one or more structure-based docking tools, wherein the generating of the at least one docking score comprises the steps of:
selecting a receptor structure file;
generating docking poses and associated binding affinity scores for each of the one or more ligand-protein complexes by one or more docking tools; analyzing the resulting docking poses for the one or more ligand-protein complexes; and
generating at least one deep learning (DL) score for the one or more ligand-protein complexes using one or more sequence-based DL models, wherein the generating of the at least one deep learning score comprises the steps of:
selecting at least one DL model to predict binding affinities of ligand-protein complexes; and
generating scores for the one or more ligand-protein complexes using the at least one DL model;
generating a meta-model training dataset from scores by at least two of docking tools or DL models and at least one physicochemical property of the ligand or the protein; and
training a machine learning meta-model on the training dataset to generate an algorithm for predicting ligand-protein binding affinities.

2. The method of claim 1, wherein the generating at least one docking score for the binding affinity comprises employing at least two empirical scoring functions (ESFs) in tandem.

3. The method of claim 2, wherein the at least two ESFs are SMINA and Vinardo.

4. The method of claim 1, further comprising preprocessing the docking scores for the one or more ligand poses in predicting each ligand-protein complex.

5. The method of claim 4, wherein the preprocessing comprises filtering the ligand poses based upon the experimental and/or predicted docking poses.

6. The method of claim 5, wherein the filtering comprises applying a root-mean-squared deviation (RMSD) filter.

7. The method of claim 5, wherein the filtering comprises at least one of experimental pose filtering or consensus pose filtering.

8. The method of claim 7, wherein the experimental pose filtering comprises calculating a deviation for at least one of the docking poses predicted by the ESF relative to an experimental structure of the ligand.

9. The method of claim 7, wherein the consensus pose filtering comprises calculating a deviation between the docking poses predicted by one of the ESFs relative to the docking pose predicted by another one of the ESFs.

10. The method of claim 5, wherein the filtering cut-off is about 3 Angstrom.

11. The method of claim 1, wherein selecting the at least one DL model comprises:
inputting ligand-encoding and receptor-encoding embeddings into one or more DL models;
determining a performance of the one or more DL models; and
selecting the at least one DL model based upon the performance of the one or more DL models.

12. The method of claim 11, wherein the at least one DL model includes at least one of a pre-trained model or a de novo-trained model.

13. The method of claim 12, wherein the at least one DL model is either pre-trained or de novo-trained using BindingDB.

14. The method of claim 13, wherein the at least one DL model is selected from the group consisting of CNN-CNN, Daylight-AAC, Morgan-AAC, Morgan-CNN, MPNN-CNN, Transformer-CNN, MPNN-Quasi-seq, CNN/RNN-CNN/RNN, CNN-AAC, MPNN-Transformer, MPNN-PseudoAAC, MPNN-AAC, and MPNN-CNN/RNN.

15. The method of claim 13, wherein the at least one pre-trained DL model is selected from the group consisting of CNN-CNN, Daylight-AAC, Morgan-AAC, Morgan-CNN, MPNN-CNN, and Transformer-CNN.

16. The method of claim 13, wherein the at least one de novo-trained DL model is selected from the group consisting of CNN-AAC, MPNN-CNN, CNN-CNN, Daylight-CNN, MPNN-AAC, and MPNN-Transformer.

17. The method of claim 13, wherein the at least one pre-trained or the de-novo-trained model is fine-tuned using PDBbind data.

18. The method of claim 12, wherein the at least one DL model is de novo-trained using PDBbind data.

19. The method of claim 18, wherein the at least one de novo-trained DL model is selected from the group consisting of CNN-AAC, MPNN-CNN, CNN-CNN, Daylight-CNN, MPNN-AAC, MPNN-Transformer, Daylight-AAC, Morgan-AAC, Morgan-CNN, and Transformer-CNN.

20. The method of claim 11, wherein the at least one DL model is either de novo-trained or fine-tuned using 10 repetitions of 5-fold cross-validations (CVs).

21. The method of claim 20, wherein principal component analysis (PCA) is performed on the DL scores from all CVs.

22. The method of claim 21, wherein prediction scores are generated from the first principal components of the PCA.

23. The method of claim 11, wherein the ligand-encoding and the receptor-encoding embeddings is from DeepPurpose library.

24. The method of claim 11, wherein the selecting comprises selecting the DL model that achieves the performance of mean-squared-error (MSE)<0.6.

25. The method of claim 1, wherein the meta-model is a linear model or a non-linear model and is selected from the group consisting of LASSO model, ElasticNet model, Linear Regression model, and XGBoost model.

26. The method of claim 1, wherein the meta-model training dataset further includes molecular weight of the at least two or more ligands.

27. An apparatus for predicting a ligand-protein binding affinity, the apparatus comprising:
a processor;
a memory unit; and
a communication interface;
wherein the processor is connected to the memory unit and the communication interface; and
wherein the processor and memory are configured to implement the algorithm trained according to claim 1.

28. A non-transitory computer readable storage medium, storing computer-executable instructions for performing the method of claim 1.
